SEQUENCE LISTING (38 sequences)

SEQ ID NO: 1 - protein, 37 aa, Homo sapiens
  misc_feature: Search sequence
IPELELPSSSPVPQVTTPEPGIYHGICLQLNFTASWG

SEQ ID NO: 2 - protein, 1669 aa, Homo sapiens
MPKTHGAIRQSLGLQVAGAMRLGCWAFVKGYAKDMVTDFDEKHDEYLILLQQRNRILKHLKSKDPVQLRLEHLEQGFSVYVNGANSELKSSPRKAIHSDFSRSASHTEGTHDYGRRTLFREAEEALRRSSRTAPSKVQRRGWHQKSVQIRTEAGPRLHIEPPVDYSDDFELCGDVTLQANNTSEDRPQDTKSPTSTKPCGFNFSLLNACGHSDPSSSVDKERILLTIQDVMELRRSLELSVNLQRKQKDCSSDEYDSIEEDILSEPEPEDPALVGHPRHDRPPSSGDWTQKDVHGEQETEGRSSPGPDTLVVLEFNPASKSHKRERNLSAKRKDNAEVFVPTKPEPNLTPQAPAVFPDQERMCSRPGSRRERPLSATRKTLCEAEYPEEDASAVLQAIQVENAALQRALLSRKAEQPASPLQDAEGPPAKPWTSLLEEKEETLELLPITTATTTQEPAGAAGGARAINQAMDRIGLLGSRQQQKLLKVLQAVESDSAHLGRVVSPTKEQVSDTEDKQRMRADEIKDAIYVTMEILSNWGNSWWVGLTEVEFFDLNDTKLYVSPHDVDIRNTATPGELGRLVNRNLAGKKDSSPWTCPFHPPLQLFFVIRNTRQLGDFHLAKIKVRNYWTADGDLDIGAKNVKLYVNRNLIFNGKLDKGDREAPADHSILVDQKNEKSEQLEEAMNAHSEESKGTHEMAGASGDKELGLGCSPPAETLADAKLSSQGNVSGKRKNSTNCRKDSLSQLEEYLRLSAVPTSMGDMPSAPATSPPVKCPPVHEEPSLIQQLENLMGRKICEPPGKTPSWLQPSPTGKDRKQGGRKPKPLWLSPEKPLAWKGRLPSDDVIGEGPGETEARDKGLRHEPGWGTSRSVNTKERPQRATTKVHSDDSDIFNQPPNRERPASGRRGSRKDAGSSSHGDDQPASREDTWSSRTPSRSRWRSEQEHTLHESWSSLSAFDRSHRGRISNTELPGDILDELLQQKSSRHSDLPPSKKGEQPGLSRGQDGYSGETDAGGDFKIPVLPYGQRLVIDIKSTWGDRHYVGLNGIEIFSSKGEPVQISNIKADPPDINILPAYGKDPRVVTNLIDGVNRTQDDMHVWLAPFTRGRSHSITIDFTHPCHVALIRIWNYNKSRIHSFRGVKDITMLLDTQCIFEGEIAKASGTLAGAPEHFGDTILFTTDDDILEAIFYSDEMFDLDVGSLDSLQDEEAMRRPSTADGEGDERPFTQAGLGADERIPELELPSSSPVPQVTTPEPGIYHGICLQLNFTASWGDLHYLGLTGLEVVGKEGQALPIHLHQISASPRDLNELPEYSDDSRALDKLIDGTNITMEDEHMWLIPFSPGLDHVVTIRLDRAESIAGLRFWNYNKSPEDTYRGAKIVHVSLDGLCVSPPEGFLIRKGPGNCHFDFAQEILFVDYLRAQLLPQPARRLDMRSLECASMDYEAPLMPCGFIFQFQLLTSWGDPYYIGLTGLELYDERGEKIPLSENNIAAFPDSVNSLEGVGGDVRTPDKLIDQVNDTSDGRHMWLAPILPGLVNRVYVIFDLPTTVSMIKLWNYAKTPHRGVKEFGLLVDDLLVYNGILAMVSHLVGGILPTCEPTVPYHTILFTEDRDIRHQEKHTTISNQAEDQDVQMMNENQIITNAKRKQSVVDPALRPKTCISEKETRRRRC

SEQ ID NO: 3 - protein, 1081 aa, Homo sapiens
KDSSPWTCPFHPPLQLFFVIRNTRQLGDFHLAKIKVRNYWTADGDLDIGAKNVKLYVNRNLTFNGKLDKGDREAPADHSILVDQKNEKSEQLEEAMNAHSEESKGTHEMAGASGDKELGLGCSPPAETLADAKLSSQGNVSGKRKNSTNCRKDSLSQLEEYLRLSAVPTSMGDMPSAPATSPPVKCPPVHEEPSLIQQLENLMGRKICEPPGKTPSWLQPSPTGKDRKQGGRKPKPLWLSPEKPLAWKGRLPSDDVIGEGPGETEARDKGLRHEPGWGTSRSVNTKERPQRATTKVHSDDSDIFNQPPNRERPASGRRGSRKDAGSSSHGDDQPASREDTWSSRTPSRSRWRSEQEHTLHESWSSLSAFDRSHRGRISNTELPGDILDELLQQKSSRHSDLPPSKKGEQPGLSRGQDGYSGETDAGGDFKIPVLPYGQRLVIDIKSTWGDRHYVGLNGIEIFSSKGEPVQISNIKADPPDINILPAYGKDPRVVTNLIDGVNRTQDDMHVWLAPFTRGRSHSITIDFTHPCHVALIRIWNYNKSRIHSFRGVKDITMLLDTQCIFEGEIAKASGTLAGAPEHFGDTILFTTDDDILEAIFYSDEMFDLDVGSLDSLQDEEAMRRPSTADGEGDERPFTQAGLGADERIPELELPSSSPVPQVTTPEPGIYHGICLQLNFTASWGDLHYLGLTGLEVVGKEGQALPIHLHQISASPRDLNELPEYSDDSRTLDKLIDGTNITMEDEHMWLIPFSPGLDHVVTIRLDRAESIAGLRFWNYNKSPEDTYRGAKIVHVSLDGLCVSPPEGFLIRKGPGNCHFDFAQEILFVDYLRAQLLPQPARRLDMRSLECASMDYEAPLMPCGFIFQFQLLTSWGDPYYIGLTGLELYDERGEKIPLSENNIAAFPDSVNSLEGVGGDVRTPDKLIDQVNDTSDGRHMWLAPILPGLVNRVYVIFDLPTTVSMIKLWNYAKTPHRGVKEFGLLVDDLLVYNGILAMVSHLVGGILPTCEPTVPYHTILFTEDRDIRHQEKHTTISNQAEDQDVQMMNENQIITNAKRKQSVVDPALRPKTCISEKETRRRRC

SEQ ID NO: 4 - protein, 1625 aa, Homo sapiens
PEPPPLGMDGQTLRKAERSWSCSREKKEGYAKDMVTDFDEKHDEYLILLQQRNRILKHLKSKDPVQLRLEHLEQGFSVYVNGANSELKSSPRKAIHSDFSRSASHTEGTHDYGRRTLFREAEEALRRSSRTAPSKVQRRGWHQKSVQIRTEAGPRLHIEPPVDYSDDFELCGDVTLQANNTSEDRPQELRRSLELSVNLQRKQKDCSSDEYDSIEEDILSEPEPEDPALVGHPRHDRPPSSGDWTQKDVHGEQETEGRSSPGPDTLVVLEFNPASKSHKRERNLSAKRKDNAEVFVPTKPEPNLTPQAPAVFPDQERMCSRPGSRRERPLSATRKTLCEAEYPEEDASAVLQAIQVENAALQRALLSRKAEQPASPLQDAEGPPAKPWTSLLEEKEETLELLPITTATTTQEPAGAAGGARAINQAMDRIGLLGSRQQQKLLKVLQAVESDSAHLGRVVSPTKEQVSDTEDKQRMRADEIKDAIYVTMEILSNWGNSWWVGLTEVEFFDLNDTKLYVSPHDVDIRNTATPGELGRLVNRNLAGKKDSSPWTCPFHPPLQLFFVIRNTRQLGDFHLAKIKVRNYWTADGDLDIGAKNVKLYVNRNLIFNGKLDKGDREAPADHSILVDQKNEKSEQLEEAMNAHSEESKGTHEMAGASGDKELGLGCSPPAETLADAKLSSQGNVSGKRKNSTNCRKDSLSQLEEYLRLSAVPTSMGDMPSAPATSPPVKCPPVHEEPSLIQQLENLMGRKICEPPGKTPSWLQPSPTGKDRKQGGRKPKPLWLSPEKPLAWKGRLPSDDVIGEGPGETEARDKGLRHEPGWGTSRSVNTKERPQRATTKVHSDDSDIFNQPPNRERPASGRRGSRKDAGSSSHGDDQPASREDTWSSRTPSRSRWRSEQEHTLHESWSSLSAFDRSHRGRISNTELPGDILDELLQQKSSRHSDLPPSKKGEQPGLSRGQDGYSGETDAGGDFKIPVLPYGQRLVIDIKSTWGDRHYVGLNGIEIFSSKGEPVQISNIKADPPDINILPAYGKDPRVVTNLIDGVNRTQDDMHVWLAPFTRGRSHSITIDFTHPCHVALIRIWNYNKSRIHSFRGVKDITMLLDTQCIFEGEIAKASGTLAGAPEHFGDTILFTTDDDILEAIFYSDEMFDLDVGSLDSLQDEEAMRRPSTADGEGDERPFTQAGLGADERIPELELPSSSPVPQVTTPEPGIYHGICLQLNFTASWGDLHYLGLTGLEVVGKEGQALPIHLHQISASPRDLNELPEYSDDSRTLDKLIDGTNITMEDEHMWLIPFSPGLDHVVTIRLDRAESIAGLRFWNYNKSPEDTYRGAKIVHVSLDGLCVSPPEGFLIRKGPGNCHFDFAQEILFVDYLRAQLLPQPARRLDMRSLECASMDYEAPLMPCGFIFQFQLLTSWGDPYYIGLTGLELYDERGEKIPLSENNIAAFPDSVNSLEGVGGDVRTPDKLIDQVNDTSDGRHMWLAPILPGLVNRVYVIFDLPTTVSMIKLWNYAKTPHRGVKEFGLLVDDLLVYNGILAMVSHLVGGILPTCEPTVPYHTILFTEDRDIRHQEKHTTISNQAEDQDVQMMNENQIITNAKRKQSVVDPALRPKTCISEKETRRRRC

SEQ ID NO: 5 - protein, 965 aa, Mus musculus
GPFALKTGRNVSNSSHPLAFQSKKDPPVWTCPFHPPLQLYFIIHNMRQLRDFGLTMIKVRNYWTADGDLDIGAKNVKLYVNKSLIFDGVLEKGGGEAPSDCTIPVDLQRERNESSDKALSTGWKESKGALKMAALSDARELGLSCSQPAESLDMTVSSQGDFLGEKVNSTSGLKNSLSKLQEDVRLLATPASMGDGPSVPSSSSPGKCLPLEEEPSLIQQLESLREDTQASQTLPWLQWYGEQEHALHASWDSLTAFDRAHRGRISALEPQGDILDEFLKQQRSSRHEEFPAPCREEEPEPSTEMGGDSDFKIPVLPYGQHLVIDIKSTWGDRHYVGLNGIEIFSSSGEPVQISSITADPPDINILPAYGKDPRVVSNLIDGVNRTQDDMHVWLAPFTPGMTHTISIEFTHPCQVALIRIWNYNKSRIHSFRGVKDITMLLDTQCIFEGEIAKASGTLMGAPEHFGDTILFTMDEDILEAIFCLDDTFDMDAESLCGLQPEEALRRPSTADGEGQDERPFTQAGLGAQDQVPGLELQTSPPVSEVTTPEPGIFYGLCLRLNLTASWGDLHYIGLTGLEVVGKDGEALPIQPHQLSASPRDLNDLPEYNDDSRTLDKLIDGMNITTEDEHMWLIPFSPGLDHVVMIHFDRAQSIAGLRLWNYNKSPEDTYRGVKIAHVSLDGLCVSPAEGFLIRKGPGNCHFDFAQEILFGDYLQTRLPPAPTRRLDAKSLERASMDYEAPLMPCGFIFQFQLLSSWGDPYYIGLTGLELYDEHGERIPLSQNNIAAFPDSVNALEGVCGDVRTPDKLIDQVNDTSDGRHMWLAPILPGLVNRVYVIFDLPTTVSMIKLWNYTKTPQRGVKEFGLLVDDLLVYNGILAMVSHLVGGILPTCEPTVPHHTILFAEDTDFCHQEKHAIISKPEEDQDIQMMNENQVITTSRRKPGTADPALRPKTCIREKETSRRWRC

SEQ ID NO: 6 - protein, 661 aa, Mus musculus
MGGDSDFKIPVLPYGQHLVIDIKSTWGDRHYVGLNGIEIFSSSGEPVQISSITADPPDINILPAYGKDPRVVSNLIDGVNRTQDDMHVWLAPFTPGMTHTISIEFTHPCQVALIRIWNYNKSRIHSFRGVKDITMLLDTQCIFEGEIAKASGTLMGAPEHFGDTILFTMDEDILEAIFCLDDTFDMDAESLCGLQPEEALRRPSTADGEGQDERPFTQAGLGAQDQVPGLELQTSPPVSEVTTPEPGIFYGLCLRLNLTASWGDLHYIGLTGLEVVGKDGEALPIQPHQLSASPRDLNDLPEYNDDSRTLDKLIDGMNITTEDEHMWLIPFSPGLDHVVMIHFDRAQSIAGLRLWNYNKSPEDTYRGVKIAHVSLDGLCVSPAEGFLIRKGPGNCHFDFAQEILFGDYLQTRLPPAPTRRLDAKSLERASMDYEAPLMPCGFIFQFQLLSSWGDPYYIGLTGLELYDEHGERIPLSQNNIAAFPDSVNALEGVCGDVRTPDKLIDQVNDTSDGRHMWLAPILPGLVNRVYVIFDLPTTVSMIKLWNYTKTPQRGVKEFGLLVDDLLVYNGILAMVSHLVGGILPTCEPTVPHHTILFAEDTDFCHQEKHAIISKPEEDQDIQMMNENQVITTSRRKPGTADPALRPKTCIREKETSRRWRC

SEQ ID NO: 7 - protein, 532 aa, Mus musculus
DITMLLDTQCIFQGEIAKASGTLMGAPEHFGDTILFTMDEDILEAIFCLDDTFDMDAESLCGLQPEEALRRPSTADGEGQDERPFTQAGLGAQDQVPGLELQTSPPVSEVTTPEPGIFYGLCLRLNLTASWGDLHYIGLTGLEVVGKDGEALPIQPHQLSASPRDLNDLPEYNDDSRTLDKLIDGMNITTEDEHMWLIPFSPGLDHVVMIHFDRAQSIAGLRLWNYNKSPEDTYRGVKIAHVSLDGLCVSPAEGFLIRKGPGNCHFDFAQEILFGDYLQTRLPPAPTRRLDAKSLERASMDYEAPLMPCGFIFQFQLLSSWGDPYYIGLTGLELYDEHGERIPLSQNNIAAFPDSVNALEGVCGDVRTPDKLIDQVNDTSDGRHMWLAPILPGLVPKLPTLQRSLGLGRVWEAGRVRGAGLTGAGPTLSQLLVDDLLVYNGILAMVSHLVGGILPTCEPTVPHHTILFAEDTDFCHQEKHAIISKPEEDQDIQMMNENQVITTSRRKPGTADPALRPKTCIREKETSRRWRC

SEQ ID NO: 8 - protein, 1019 aa, Mus musculus
MEFNPASKGNKMDRILSAKRKENAEVFIPSKPDSVLNPQPPAVFPEQERACSRSGSRRDRPLSATRKAYAAEDREEDASAVLKAIQVENEALQQVFLSHDPELHTSPQQDTKEPPAKSWSSLLKAKEDIPELLPATPVTTDPELCRAAAGAKAVSQAMDGMSPLGSRQQHKLVGVLQTMESDTTHLSQVAIPTEKPVPNSEEKWKARVDEIEDAIYVTMEILSNWGNASWVGLTEVQFFDLNNIKLYVSPHDVDIRNAVLPGELGCLVNRDLVSKKDPPVWTCPFHPPLQLYFIIHNMRQLRDFGLTMIKVRNYWTADGDLDIGAKNVKLYVNKSLIFDGVLEKGGGEAPSDCTIPVDLQRERNESSDKALSTGWKESKGALKMAALSDARELGLSCSQPAESLDMTVSSQGDFLGEKVNSTSGLKNSLSKLQEDVRLLATPASMGDGPSVPSSSSPGKCLPLEEEPSLIQQLESLRGRKIPEPTGKTPHWLQPSLAGMGKKQTVRKPKPLWLSPEKDLEQKSRFPSEDVMGDTPGEVETREKGPRREQGRTSSWNVITEERAPKAFSKACGDDLDIFSQLPNRDRPASGRRALKKEASSSHGDDRPASKEDTQASQTLPWLQWYGEQEHALHASWDSLTAFDRAHRGRISALEPQGDILDEFLKQQRSSRHEEFPAPCREEEPEPSTEMGGDSDFKIPVLPYGQHLVIDIKSTWGDRHYVGLNGIEIFSSSGEPVQISSITADPPDINILPAYGKDPRVVSNLIDGVNRTQDDMHVWLAPFTPGMTHTISIEFTHPCQVALIRIWNYNKSRIHSFRGVKDITMLLDTQCIFEGEIAKASGTLMGAPEHFGDTILFTMDEDILEAIFCLDDTFDMDAESLCGLQPEEALRRPSTADGEGQDERPFTQAGLGAQDQVPGLELQTSPPVSEVTTPEPGIFYGLCLRLNLTASWGDLHYIGLTGLEVVGKDGEALPIQPHQLSASPRDLNDLPEYNDDSRTLDKCPFTVALSPAGTDCLGSP

SEQ ID NO: 9 - protein, 372 aa, Rattus norvegicus
MGIVATARVAHSTDVLKVLRVQGLIDGMNITTEDEHMWLIPFSPGLDHVVMIHFDRAQSIAGLRLWNYNKSPEDTYRGVKIAHVSLDGLCVSPAEGFLIRKGPGNCHFDFAQEILFGDYLQTRLPPAPSRRLDAKSLERASMDYEAPLMPCGFIFQFQLLSSWGDPYYIGLTGLELYDEHGERIPLSQNNIAAFPDSVNALEGVCGDVRTPDKLIDQVNDTSDGRHMWLAPILPGLVNRVYVIFDLPTTVSMIKLWNYTKTPQRGVKEFGLLVDDLLVYNGILAMVSHVVGGILPTCEPTVPHHTILFAEDTDLCHQEKHTIISKPEEDQDIQMMNENQVITTSRRKPATADPALRPKTCIREKESSRRWRC

SEQ ID NO: 10 - protein, 40 aa, Artificial
  Search template
IPELELPSSSPVPQVTTPEPGIYHGICLZLNFTASWGDLH

SEQ ID NO: 11 - protein, 40 aa, Homo sapiens
IPELELPSSSPVPQVTTPEPGIYHGICLQLNFTASWGDLH

SEQ ID NO: 12 - protein, 40 aa, Mus musculus
VPGLELQTSPPVSEVTTPEPGIFYGLCLRLNLTASWGDLH

SEQ ID NO: 13 - protein, 40 aa, Rattus norvegicus
VPGLELQSGPPVSDVFTPEPGIFYGLCLRLNFTASWGDLH

SEQ ID NO: 14 - protein, 9 aa, Homo sapiens
ELNFTASWG

SEQ ID NO: 15 - protein, 9 aa, Rattus norvegicus
RLNFTASWG

SEQ ID NO: 16 - protein, 9 aa, Mus musculus
RLNLTASWG

SEQ ID NO: 17 - protein, 8 aa, Schistocerca gregaria
  MOD_RES (1)..(1): PYRROLIDONE CARBOXYLIC ACID
ELNFSTGW

SEQ ID NO: 18 - protein, 10 aa, Locusta migratoria
  MOD_RES (1)..(1): PYRROLIDONE CARBOXYLIC ACID
ELNFTPNWGT

SEQ ID NO: 19 - protein, 10 aa, Blaberus discoidalis
  MOD_RES (1)..(1): PYRROLIDONE CARBOXYLIC ACID
EVNFSPGWGT

SEQ ID NO: 20 - protein, 10 aa, Artificial
  Synthetic HTP analog
EVNFSPAWGT

SEQ ID NO: 21 - protein, 10 aa, Artificial
  Synthetic HTP analog
EVNFSPGAGT

SEQ ID NO: 22 - protein, 10 aa, Artificial
  Synthetic HTP analog
EVNFSPGWAT

SEQ ID NO: 23 - protein, 9 aa, Manduca sexta
  MOD_RES (1)..(1): PYRROLIDONE CARBOXYLIC ACID
ELTFTSSWG

SEQ ID NO: 24 - protein, 8 aa, Homo sapiens
ELNFTASW

SEQ ID NO: 25 - protein, 9 aa, Homo sapiens
  MOD_RES (1)..(1): PYRROLIDONE CARBOXYLIC ACID
ELNFTASWG

SEQ ID NO: 26 - protein, 9 aa, Homo sapiens
  MOD_RES (9)..(9): AMIDATION
ELNFTASWG

SEQ ID NO: 27 - protein, 8 aa, Homo sapiens
  MOD_RES (1)..(1): PYRROLIDONE CARBOXYLIC ACID
ELNFTASW

SEQ ID NO: 28 - protein, 8 aa, Homo sapiens
  MOD_RES (8)..(8): AMIDATION
ELNFTASW

SEQ ID NO: 29 - protein, 9 aa, Homo sapiens
  MOD_RES (1)..(1): PYRROLIDONE CARBOXYLIC ACID
ELNFTASWG

SEQ ID NO: 30 - protein, 8 aa, Homo sapiens
  MOD_RES (1)..(1): PYRROLIDONE CARBOXYLIC ACID
ELNFTASW

SEQ ID NO: 31 - protein, 11 aa, artificial
  Claim 1, claim 9
XXXXXXXXXXX

SEQ ID NO: 32 - protein, 11 aa, Artificial
  Claim 2, claim 10
XXXXXXXXXXX

SEQ ID NO: 33 - protein, 11 aa, Artificial
  Claim 3, claim 11
XXXXXXXXXXX

SEQ ID NO: 34 - protein, 11 aa, artificial
  Claim 4, claim 12
XXXXXXXXXXX

SEQ ID NO: 35 - protein, 11 aa, Artificial
  Claim 5, claim 13
XXXXXXXXXXX

SEQ ID NO: 36 - protein, 11 aa, Artificial
  Claim 6, claim 14
XXXXXXXXXXX

SEQ ID NO: 37 - protein, 11 aa, Artificial
  Claim 15
XXXXXXXXXXX

SEQ ID NO: 38 - protein, 11 aa, Artificial
  Claim 22
XLNXTASWXXX

(19) United States
(12) Patent Application Publication
(10) Pub. No.: US 2005/0222040 A1
(43) Pub. Date: Oct. 6, 2005

(54) Vertebrate peptide modulators of lipid metabolism

(75) Inventors: Bernice Z. Schacter, Wilmington, DE (US); Lee P. Schacter, Wilmington, DE (US); Michael H. Zeldin, Cambridge, MA (US)

Correspondence Address: GODFREY & KAHN S.C., 780 NORTH WATER STREET, MILWAUKEE, WI, 53202, US

(73) Assignee: BLM Group, Inc., Cambridge, MA (US)

(21) Appl. No.: 10/818,036

(22) Filed: Apr. 5, 2004

(51) Int. Cl. A61K 38/08; C07K 7/06

(52) U.S. Cl. 514/15; 530/328

(57) ABSTRACT

The invention provides vertebrate lipid mobilizing peptides, derivative compounds and compositions as well as methods of using such peptides, derivative compounds and compositions for modulating the lipid metabolism of a vertebrate subject.

### BACKGROUND OF THE INVENTION

Obesity and less severe overweight conditions are a significant cause of morbidity and mortality in humans. High body weight is a risk factor for many diseases and disorders, particularly when fat comprises a high percentage of body weight. For example, incidence of each of type II diabetes, cholelithiasis, hypertension, and coronary heart disease is much greater in obese humans than in non-obese humans. Other diseases associated with obesity include arthritis, various cancers (e.g., breast, colorectal, and endometrial cancers), renal failure, liver disease, chronic pain (e.g., lower back pain), sleep apnea, stroke, and urinary incontinence.

In addition to medical risks attributable to large amounts of body fat, fat accumulation is considered by many to be cosmetically undesirable as well. Likely attributable to popular notions regarding desirable body size and shape, many people are afflicted by psychological disruptions that might be alleviated if body fat were reduced or more easily controlled.

Body mass index (BMI) is a common measurement used to diagnose overweight and obesity. BMI is calculated by dividing an individual's weight in kilograms by the square of the individual's height in meters. Weight classifications have been developed by the National Heart, Lung, and Blood Institute (NHLBI), and these classifications can be used to divide the population into six groups, based on BMI, as follows:

TABLE 1

| | BMI |
|---|---|
| | (body weight in kilograms/ |
| Classification | height in meters squared) |
| Underweight | <18.5 |
| Normal | 18.5 to <25.0 |
| Overweight | 25.0 to <30.0 |
| Obesity Class 1 | 30.0 to <35.0 |
| Obesity Class 2 | 35.0 to <40.0 |
| Obesity Class 3 | >40.0 |

Using the NHLBI criteria, 17.9% of the U.S. population was obese (obesity class 1, 2, or 3) in 1998, corresponding to more than 45 million individuals. Estimates of medical costs attributable to obesity and related conditions were about $100 billion in the United States in 1999 (American Obesity Association report, 1999, “Costs of Obesity”). Furthermore, significant costs are associated with weight loss programs undertaken by individuals (e.g., about $33 billion per year in the U.S. in the late 1990s; 1998 Federal Trade Commission Report, “Consumer Weight Loss Products and Programs”).

In general, obesity is a disease of affluence. Industrialization and increased national wealth are associated with a shift of the percent of people with BMI's <25 to those with BMI's >25. This shift is roughly proportional to per capita income and has occurred in all developed and developing nations from Europe to North America to Asia. This suggests, that when food resources are not limiting, the natural tendency of the species is to achieve a high BMI and that the problem of obesity is unlikely to respond to public health measures, education and diet. Successful treatment will require pharmacological intervention.

Using a body mass index >30 kg/m² as the definition of obesity, the percent of the U.S. population considered medically obese in 1998 was 17.9%, up from 12.0% in 1991. For a population of 250,000,000 this would consist of 44,750,000 individuals.

Estimates for obesity in Europe are comparable and show a similar trend. In England about 5% of people had a BMI >30 kg/m² in 1980, while in 1995 about 15% had achieved that mass. The increase has been much less dramatic in Holland but even so about 7% were considered obese in 1995. Almost 20% of former East Germans were obese in 1992. Overall data would suggest that at least 15% of the North American and Western European population could medically benefit from weight loss or about 75,000,000 individuals, assuming an overall population of 500,000,000. If one includes those individuals classified as “overweight” the prevalence may approach 50% of the population. The need can only be expected to grow as affluence increases in Asia.

Medical Risks and Costs of Obesity

The relative risk of Type II diabetes is increased six-fold for a BMI of about 25 kg/m². The relative risk of cholelithiasis, hypertension and coronary heart disease are increased three-fold for a BMI of 30 kg/m². Other diseases considered associated with varying degrees of certainty with obesity include; arthritis, breast cancer, colorectal cancer, endometrial cancer, renal failure, liver disease, low back pain, sleep apnea, stroke and urinary incontinence. In 1999, the medical costs of the diseases related to obesity were about $100 billion. Most of these costs are associated with heart disease, stroke, type II diabetes, hypertension and arthritis.

At this time treatment of obesity takes four forms; diet, pharmaceuticals, surgery and herbs. Diet, either ad hoc or using planned meals, has a very low rate of long-term benefit. One summary of multiple clinical trials showed a 15 kg weight loss at 6 months but return to baseline by 5 years in the vast majority of subjects. As stated in 1958 by Stunkard: “Most obese persons will not stay in treatment for obesity. Of those who stay in treatment, most will not lose weight, and of those who do lose weight, most will regain it” (Stunkard, A. J., The management of obesity. NY State J Med 58: 79-87, 1958). This situation has remained unchanged for over 40 years.

Pharmaceuticals have had a mixed history for the treatment of obesity. Multiple agents have been tried and many withdrawn or restricted due to toxicity. None have proven especially successful in terms of weight control or especially profitable. Amphetamines are early examples of active agents with toxicity and abuse issues. The beta adrenergic agents continue to be available but lack of selectivity creates significant toxicity in addition to misuse potential. The problems with FenPhen (Fenfluramine and Phenteramine) show both the problems inherent with relatively non-specific agents and the safety concerns for successful use of a drug by as much as one-third of the population.

At this time, three classes of drugs are available to promote weight loss; noradrenergic agents, serotonergic agents, and lipase inhibitors to block GI absorption of fats (Table 2).

TABLE 2
DRUGS FOR WEIGHT CONTROL

| DRUG | TRADE NAME(s) |
|---|---|
| Noradrenergic Agents | |
| Benzphetamine | Didrex |
| Phendimetrazine | Anorex; Obalan; Phendiet; Plegine; Wehless; etc. |
| Diethylpropion | Tenuate; Tepanol |
| Mazindol | Mazanor; Sanorex |
| Phenteramine | Fastin; Ionamin; Phentrol; Adipex-P; etc. |
| Phenylpropanolamine | Dexatrim |
| Serotonergic Agents | |
| Fenfluramine | Pondimin |
| Fluoxetine | Prozac; Lovan |

Serotonergic and Noradrenergic Agents

| | |
|---|---|
| Sibutramine | Meridia |
| Lipase Inhibitor | |
| Orlistat | Xenical |

The efficacy of herbals and other nutraceuticals for the treatment of obesity has not been scientifically demonstrated. A nutraceutical, originally defined as a substance that is a food or a part of a food that provides medical or health benefits including prevention and treatment of disease, has now been defined as a product isolated or purified from foods, generally sold in medicinal forms not usually associated with food, and demonstrated to have a physiological benefit or provide protection against chronic disease. While such agents are probably not effective, their sales are driven by advertisements and hope and are unlikely to be affected by a lack of credible clinical data.

Surgical treatment for obesity has taken a variety of forms. At this time, two types of operations are popular; vertical gastroplasty with artificial pseudopylorus (VGAP) and Roux-en-Y gastric resection. Many, if not most, of these procedures are done laparascopically. Given the complications of surgery, its variable results, the costs, and the number of candidates for the procedure, surgical treatment of obesity is unlikely to become routine or common. Significant economic, medical, and psychological gains could be achieved if compositions and methods could be developed that allow people to lose weight.

In summary, prior art weight loss methods and compositions have not been widely successful. Current treatments for obesity and overweight include diet, pharmaceutical agents, surgery, and herbal therapy. Dietary methods for inhibiting or reversing obesity and overweight have a very low long-term benefit rate. Although some pharmaceutical agents (and combinations of agents) have exhibited the ability to reduce body weight, many of these agents have been withdrawn from markets owing to toxicity, lack of efficacy, or both. Surgical methods of treating obesity and overweight are costly, are sometimes accompanied by very serious complications, exhibit significant variation in outcome, and are not amenable for use in all patients. Herbal and nutraceutical compositions for weight loss are popular, but their efficacy is typically not demonstrated. Owing to their often unknown mechanism of action, the variability of their composition, and their lack of credible clinical data, herbal weight loss compositions are not suitable for widespread use in the population.

A critical need remains for compositions and methods that can be used to effect weight loss in humans. The present invention satisfies this need, at least in part, by providing such compositions and methods.

### SUMMARY OF THE INVENTION

In preferred embodiments, the invention provides vertebrate lipid-mobilizing peptides (VLMPs) useful for mobilizing lipids from cells such as adipocytes when the cells are contacted with at least one vertebrate lipid-mobilizing peptide. The VLMP can be an isolated native peptide, a synthetic peptide or a semi-synthetic peptide. In other embodiments, the invention provides compositions comprising a therapeutically effective amount of a vertebrate lipid-mobilizing peptide and a pharmaceutically acceptable carrier. In further embodiments, the invention provides compositions comprising a therapeutically effective amount of a vertebrate lipid-mobilizing peptide agonist and a pharmaceutically acceptable carrier. In yet other embodiments, the invention provides compositions comprising a therapeutically effective amount of a vertebrate lipid-mobilizing peptide antagonist and a pharmaceutically acceptable carrier.

In preferred embodiments, the present invention provides an isolated vertebrate lipid mobilizing peptide encoded by a vertebrate KIAA0556 cluster gene, consisting essentially of 8-11 amino acid residues, wherein the vertebrate lipid mobilizing peptide is translated operatively linked to a secretory signal sequence, wherein the lipid metabolism of a vertebrate cell is modulated when contacted with the vertebrate lipid mobilizing peptide. In preferred embodiments, the vertebrate lipid mobilizing peptide has the structure Xaa1-Xaa2-Xaa3-Xaa4-Xaa5-Xaa6-Xaa7-Xaa8-Xaa9-Xaa10-Xaa11 (SEQ ID NO: 31), where Xaa1, Xaa2, Xaa3 and Xaa5 are any amino acid residue; Xaa4 is Phe or Leu, Xaa6 is a nonpolar amino acid residue, Xaa7 is an uncharged polar amino acid residue, Xaa9 is Gly or absent and Xaa10 and Xaa11 are present or absent. Preferably, Xaa6 is Ala and Xaa7 is Ser. Typically, Xaa2 is Leu. In preferred embodiments, Xaa3 is Asn. In other preferred embodiments, Xaa5 is Thr. Typically, Xaa8 is Trp. In some preferred embodiments, Xaa1 is selected from the group consisting of Gln, Arg, pGlu, and a pyroglutamyl alternative moiety. In other embodiments, the vertebrate lipid mobilizing peptide has a sequence selected from the group consisting of SEQ ID NOs: 31, 32, 33, 34, 35, 36, 37 and 38.

In certain preferred embodiments, least one of the amino acid residues is derivatized. When Xaa1 is a pyroglutamyl alternative moiety, it is selected from the group consisting of L-6-ketopiperidine-2-carbonyl-, (S)-4,5-dihydroorotic acid derivatives, gamma-butyrolactone-gamma-carbonyl-, L-pyro-2-aminoadipyl-, alpha-((1S,2R))-2-methyl-4-oxocyclopentylcarbonyl- and (S)-2-oxoimidazoline-4-carbonyl-derivatives.

In preferred embodiments, the isolated vertebrate lipid mobilizing peptide is naturally encoded by a vertebrate KIAA0556 cluster gene selected from the group consisting of Hs.30512, D430042O09Rik, LOC361646, Bt. 10058, Ssc.6085, Gga.9001, Xl.29814, Dr.16016 and Omy.7157. These genes are found in humans, mice, rats, cattle, pigs, chickens, frogs (Xenopus sp.) and fish (zebrafish and rainbow trout), respectively.

The present invention also provides a composition comprising a vertebrate lipid mobilizing peptide and a pharmaceutically acceptable carrier. In further preferred embodiments, the present invention provides a kit comprising a vertebrate lipid mobilizing peptide and instructions for use.

The present invention provides a method of modulating the lipid metabolism of a vertebrate subject comprising the step of administering an effective amount of a vertebrate lipid mobilizing peptide. In preferred embodiments, the present invention provides a method of reducing the body fat of a vertebrate subject comprising the step of administering an effective of a vertebrate lipid mobilizing peptide. In other preferred embodiments, the present invention provides a method of reducing the body mass index of a human subject comprising the step of administering an effective amount of a vertebrate lipid mobilizing peptide.

In preferred embodiments, such peptides and compositions are useful for mobilizing lipids in humans, including actions on human adipocytes. VLMPs and compositions comprising VLMPs can be used to modulating lipid metabolism in humans, preferably producing weight loss in humans. In preferred embodiments, VLMPs and compositions comprising VLMPs can be used to alleviate, inhibit, or reverse obesity and overweight in humans. In other embodiments, the peptides can be derivatized by known polypeptide derivatization methods and retain lipid mobilizing activity in humans. The invention includes methods, pharmaceutical compositions, kits, and screening methods relating to these compounds.

In one embodiment, the invention provides a method of promoting lipid mobilization (e.g., for the purpose of effecting weight loss, suppressing appetite, or both) in a human. The method comprises administering a VLMP to the human in an amount (e.g., 100 milligrams to 2 grams per day) effective to mobilize lipids in the human. Suitable VLMPs can promote lipid mobilization that is not significantly inhibited by propanolol.

Mobilization of lipids inhibits or prevents their storage, and promotes depletion of lipid stores. Mobilization of lipids from adipocytes increases the ability of lipids and lipid components to be taken up into the bloodstream and carried to portions of the body where they can be metabolized, transformed, or excreted. Thus, lipid mobilization permits at least partial depletion of lipid stores. Depletion of lipid stores is beneficial, for example for promoting weight loss and for enhancing metabolic availability of lipids (e.g., in humans experiencing an interruption in normal lipid metabolism.

The appetite urge experienced by humans is related in ways that are not thoroughly understood to the concentration of lipids and lipid components (e.g., fatty acids) in the bloodstream. Because the compositions and methods described herein can increase blood levels of lipids and their components, appetite can be suppressed in a human using those compositions and methods to mobilize lipids. Thus, the compositions and methods described herein can be used to decrease lipid stores, to limit appetite, or both.

In other preferred embodiments, present invention provides methods of using of VLMPs or VLMP agonists to treat obesity, diabetes, sexual dysfunction, atherosclerosis, insulin resistance, impaired glucose tolerance, hypercholesterolemia, or hypertrigylceridemia. The methods of treatment of the present invention can also include combination therapy where other pharmaceutically active compounds useful for the treatment of obesity or other diseases are used in combination with a VLMP. It is known that obese patients have higher incidences of certain diseases such as atherosclerosis, hypercholesterolemia, hypertrigylceridemia, hypertension, sexual dysfunction (including erectile dysfunction), insulin resistance, impaired glucose tolerance, diabetes, particularly non-insulin dependent diabetes mellitus (NIDDM or Type 2 diabetes) and the diseases associated with diabetes such as nephropathy, neuropathy, retinopathy, cardiomyopathy, cataracts, and polycystic ovary syndrome. These diseases can be treated indirectly by treating obesity using a VLMP or directly by treating the specific disease itself using a VLMP. These diseases can be treated in the absence of obesity using a VLMP.

In one embodiment of the invention, an obese patient or a patient at risk of becoming obese can be administered a composition comprising an active agent selected from the group consisting of a VLMP and a VLMP agonist, a pharmaceutically acceptable carrier and at least one compound useful to treat obesity, diabetes, including (NIDDM) and the conditions and/or diseases associated with diabetes, such as nephropathy, neuropathy, retinopathy, cardiomyopathy, cataracts, and polycystic ovary syndrome, atherosclerosis, hypercholesterolemia, hypertrigylceridemia, sexual dysfunction (including erectile dysfunction), insulin resistance, or impaired glucose tolerance, or combinations of compounds useful to treat these diseases.

In one embodiment, a polypeptide compound is provided to a cell by providing to the cell a nucleic acid vector comprising a nucleic acid that encodes the polypeptide operably linked with a promoter/regulatory region. When the vector is provided to the cell by a nucleic acid vector, the polypeptide compound is made by the cell by way of expression of the nucleic acid, including, optionally, the action of cellular enzymes on the resulting primary transcript (e.g., cyclization of the amino-terminal glutamate or glutamine residue). When the polypeptide compound is provided by way of a nucleic acid vector, the vector encodes a polypeptide having the chemical structure Xaa1-Xaa2-Xaa3-Xaa4-Xaa5-Xaa6-Xaa7-Xaa8-Xaa9-Xaa10-Xaa11 (SEQ ID NO: 31), where Xaa1, Xaa2, Xaa3 and Xaa5 are any amino acid residue; Xaa4 is Phe or Leu, Xaa6 is a nonpolar amino acid residue, Xaa7 is an uncharged polar amino acid residue, Xaa9 is Gly or absent and Xaa10 and Xaa11 are present or absent. Preferably, Xaa6 is Ala and Xaa7 is Ser. The nucleotide sequence used to encode the polypeptide is not critical, although it can be preferable to use codons that are efficiently expressed in the cell (codon efficiency information being available in the art). Preferably, the encoded polypeptide has the amino acid sequence of one of SEQ ID NOs: 14, 15, 16, 24, 25, 26, 27, 28 or 29. In other preferred embodiments, the encoded polypeptide has the amino acid sequence of one of SEQ ID NOs: 11, 12 or 13. In other embodiments, the encoded vertebrate lipid mobilizing peptide has a sequence selected from the group consisting of SEQ ID NOs: 31, 32, 33, 34, 35, 36, 37 and 38. The promoter/regulatory region can be one that is specifically expressed only in cells of a certain type (e.g., adipocytes). Numerous cell type-specific and other selective promoter/regulatory regions are known.

In other preferred embodiments, the invention provides kits and methods for the detection of, and preferably, the determination of the amount present, of a VLMP in a sample of tissue or bodily fluid. Generally, to detect the presence of a VLMP or fragment thereof in a patient, an aliquot (i.e., a predetermined amount) of a body fluid sample, such as urine or a vascular fluid, namely blood, plasma or serum from the patient is contacted by admixture (admixed), with an antibody composition of the present invention to form an immunoreaction admixture.

In other aspects the present invention provides for the use of VLMPs or VLMP agonists in the manufacture of medicaments for the treatment of obesity, diabetes, sexual dysfunction, atherosclerosis, insulin resistance, impaired glucose tolerance, hypercholesterolemia, or hypertrigylceridemia.

### BRIEF DESCRIPTION OF THE DRAWINGS

FIG. 1 is a graphic illustration of the results of a study showing the modulation of the lipid metabolism of human cells by synthetic peptides having the primary human amino acid sequence (peptides 52 and 53) and derivatized analogues (peptides 51, 54, 55, 60, 72 and 73), as measured by the release of glycerol from human adipocytes in vitro at three concentrations: 100 nanomolar, 1 micromolar and 10 micromolar.

### DETAILED DESCRIPTION OF THE INVENTION

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise.

“Alkyl” means the monovalent linear or branched saturated hydrocarbon radical, consisting solely of carbon and hydrogen atoms, having from one to twenty carbon atoms inclusive, unless otherwise indicated. Examples of an alkyl radical include, but are not limited to, methyl, ethyl, propyl, isopropyl, isobutyl, sec-butyl, tert-butyl, pentyl, n-hexyl, octyl, dodecyl, tetradecyl, eicosyl, and the like.

“Lower alkyl” means the monovalent linear or branched saturated hydrocarbon radical, consisting solely of carbon and hydrogen atoms, having from one to six carbon atoms inclusive, unless otherwise indicated. Examples of a lower alkyl radical include, but are not limited to, methyl, ethyl, propyl, isopropyl, tert-butyl, n-butyl, n-hexyl, and the like.

“Lower alkoxy” means the radical —O—R, wherein R is a lower alkyl radical as defined above. Examples of a lower alkoxy radical include, but are not limited to, methoxy, ethoxy, isopropoxy, and the like.

“Halogen” means the radical fluoro, bromo, chloro, and/or iodo.

“Optional” or “optionally” means that the subsequently described event or circumstance may but need not occur, and that the description includes instances where the event or circumstance occurs and instances in which it does not. For example, “optional bond” means that the bond may or may not be present, and that the description includes single, double, or triple bonds.

As used herein, “additional ingredients” can include one or more of the following: excipients, surface active agents, dispersing agents, inert diluents, granulating and disintegrating agents, binding agents, lubricating agents, sweetening agents, flavoring agents, coloring agents, preservatives, physiologically degradable compositions such as gelatin, aqueous vehicles and solvents, oily vehicles and solvents, suspending agents, dispersing or wetting agents, emulsifying agents, demulcents, buffers, salts, thickening agents, fillers, emulsifying agents, antioxidants, antibiotics, antifungal agents, stabilizing agents, and pharmaceutically acceptable polymeric or hydrophobic materials. Other “additional ingredients” which can be included in the pharmaceutical compositions of the invention are known in the art and described, for example in Gennaro, A. R., ed., Remington's The Science and Practice of Pharmacy, 20^{th} ed., Lippincott Williams & Wilkins, Philadelphia, 2003, which is incorporated herein by reference.

As used herein, “administering” or “administration” includes any means for introducing a LMP into the body, preferably into the systemic circulation. Examples include but are not limited to oral; buccal, sublingual, pulmonary, transdermal, transmucosal, as well as subcutaneous, intraperitoneal, intravenous, and intramuscular injection.

As used herein, “amino acid residue” encompasses amino acid residues that are encoded by the genetic code, amino acid residues that are not encoded by the genetic code, both D and L amino acid residues and unusual or unnatural amino acid substitutions.

As used herein, the term “antibody” (Ab) or “monoclonal antibody” (Mab) is meant to include intact molecules as well as antibody fragments (such as, for example, Fab and F(ab′)2 fragments) which are capable of specifically binding to protein. Fab and F(ab′)2 fragments lack the Fc fragment of intact antibody, clear more rapidly from the circulation, and may have less non-specific tissue binding than an intact antibody. Thus, these fragments are preferred, as well as the products of a Fab or other immunoglobulin expression library. Moreover, antibodies of the present invention include chimeric, single chain, and humanized antibodies.

An “insect adipokinetic hormone” means an adipokinetic hormone (AKH) that occurs naturally in an organism in Class Insecta, Subphylum Uniramia, and Phylum Arthropoda. Insect AKHs have chemical structures that are identical to color change hormones of various decapod crustaceans, such as the red pigment concentrating hormone isolated from prawn eye. Hence, these decapod crustacean pigment concentrating hormones are also considered insect AKHs for the purposes of this disclosure.

A “vertebrate lipid mobilizing peptide” (“VLMP”) means a vertebrate peptide capable of modulating the lipid metabolism of a vertebrate cell, e.g. a human adipocyte. The vertebrate lipid mobilizing peptide can be an isolated naturally occurring peptide, a synthetic peptide or a semi-synthetic peptide.

A “lipid mobilizing peptide receptor (“LMPR”) refers to a receptor which a lipid mobilizing peptide binds selectively, said peptide/receptor binding initiating a series of events that collectively mediate the effects of the lipid metabolism modulating peptide on the cell possessing the LMPR, such as a human adipocyte.

The phrase “lipid mobilizing peptide agonist” means a lipid mobilizing peptide receptor ligand that activates a lipid mobilizing peptide receptor.

The phrase “lipid mobilizing peptide antagonist” means a lipid mobilizing peptide receptor ligand that blocks activation of a lipid mobilizing peptide receptor.

“Lipolysis” refers to decomposition or hydrolysis of fats (i.e., lipids) into components thereof. By way of example, hydrolysis of an acyl glyceride results in cleavage of the ester bond between one or more carboxylic acid moieties of the glyceride and the glycerol moiety of the glyceride.

“Mobilization” of lipids refers to release from a lipid-containing cell (e.g., an adipocyte) of a lipid that is normally stored therein, lipolysis of the lipid, or both. Mobilization can include transfer of the lipid from the interior to the exterior of the cell in a modified form or in an unmodified form.

“Obesity,” also called corpulence or fatness, is the excessive accumulation of body fat, usually caused by the consumption of more calories than the body uses. The- excess calories are then stored as fat, or adipose tissue.

The term “obesity related disorders” includes, but is not limited to, type II diabetes, cardiovascular disease, cancer, and other disease states whose etiology stems from obesity.

An “obese” human is a human having a BMI >30.0, which includes humans classified in one of the obesity class 1, 2, and 3 categories of the NHLBI weight classification system.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked with the coding region of a gene is able to promote transcription of the coding region.

An “overweight” human is a human having a BMI >25.0 and <30.0, which includes humans classified in the overweight category of the NHLBI weight classification system.

“Subject” means mammals and non-mammals. “Mammals” means any member of the class Mammalia including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “subject” does not denote a particular age or sex.

“Pharmaceutically acceptable” means that which is useful in preparing a pharmaceutical composition that is generally safe, non-toxic, and neither biologically nor otherwise undesirable and includes that which is acceptable for veterinary as well as human pharmaceutical use.

A “pharmaceutically acceptable carrier” as used herein means a chemical composition with which a biologically active ingredient can be combined and which, following the combination, can be used to administer the active ingredient to a subject.

A “pharmaceutically acceptable” ester or salt as used herein means an ester or salt form of the active ingredient which is compatible with any other ingredients of the pharmaceutical composition and which is not deleterious to the subject to which the composition is to be administered. The terms “pharmaceutically acceptable salts” or “prodrugs” includes the salts and prodrugs of compounds that are, within the scope of sound medical judgment, suitable for use with patients without undue toxicity, irritation, allergic response, and the like, commensurate with a reasonable benefit/risk ratio, and effective for their intended use, as well as the zwitterionic forms, where possible, of the compounds.

“Pro-drug” means a pharmacologically inactive form of a compound which must be metabolized in vivo by a subject after administration into a pharmacologically active form of the compound in order to produce the desired pharmacological effect. After administration to the subject, the pharmacologically inactive form of the compound is converted in vivo under the influence of biological fluids or enzymes into a pharmacologically active form of the compound. Although metabolism occurs for many compounds primarily in the liver, almost all other tissues and organs, especially the lung, are able to carry out varying degrees of metabolism. For example, metabolism of the pro-drug may take place by hydrolysis in blood. Pro-drug forms of compounds may be utilized, for example, to improve bioavailability, mask unpleasant characteristics such as bitter taste, alter solubility for intravenous use, or to provide site-specific delivery of the compound. Reference to a compound herein includes pro-drug forms of a compound.

A discussion of the use of pro-drugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987. For example, if a compound contains a carboxylic acid functional group, a pro-drug can comprise an ester formed by the replacement of the hydrogen atom of the acid group with a group such as (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl) aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N-(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl.

Similarly, if a compound comprises an alcohol functional group, a pro-drug can be formed by the replacement of the hydrogen atom of the alcohol group with a group such as (C₁-C₆)alkanoyloxymethyl, 1-(C₁-C₆)alkanoyloxy)ethyl, 1-methyl-1-(C₁-C₆)alkan-oyloxy)ethyl, (C₁-C₆)alkoxycarbonyloxymethyl, N-(C₁-C₆)alkoxycarbonylaminomethyl, succinoyl, (C₁-C₆)alkanoyl, α-amino(C₁-C₄)alkanoyl, arylacyl and alpha-aminoacyl, or alpha-aminoacyl-alpha-aminoacyl, where each alpha-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate).

If a compound comprises an amine functional group, a pro-drug can be formed by the replacement of a hydrogen atom in the amine group with a group such as R-carbonyl, RO-carbonyl, NRR′-carbonyl where R and R′ are each independently (C₁-C₁₀)alkyl, (C₃-C₇)cycloalkyl, benzyl, or R-carbonyl is a natural alpha-aminoacyl or natural alpha-aminoacyl-, —C(OH)C(O)OY wherein Y is H, (C₁-C₆)alkyl or benzyl, —C(OY₀)Y₁ wherein Y₀ is (C₁-C₄) alkyl and Y₁ is ((C₁-C₆)alkyl, carboxy(C₁-C₆)alkyl, amino(C₁-C₄)alkyl or mono-N— or di-N,N-(C₁-C₆)alkylaminoalkyl, —C(Y₂) Y₃ wherein Y₂ is H or methyl and Y₃ is mono-N— or di-N,N-(C₁-C₆)— alkylamino, morpholino, piperidin-1-yl or pyrrolidin-1-yl.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements that are required for expression of the gene product. The promoter/regulatory sequence may, for example, be one that expresses the gene product in a constitutive, inducible, or tissue specific manner. A “constitutive” promoter is a nucleotide sequence which, when operably linked with a polynucleotide which encodes or specifies a gene product, causes the gene product to be produced in a living human cell under most or all physiological conditions of the cell. An “inducible” promoter is a nucleotide sequence which, when operably linked with a polynucleotide which encodes or specifies a gene product, causes the gene product to be produced in a living human cell substantially only when an inducer which corresponds to the promoter is present in the cell. A “tissue-specific” promoter is a nucleotide sequence which, when operably linked with a polynucleotide which encodes or specifies a gene product, causes the gene product to be produced in a living human cell substantially only if the cell is a cell of the tissue type corresponding to the promoter.

The term “salts” refers to inorganic and organic salts of compounds. These salts can be prepared in situ during the final isolation and purification of a compound, or by separately reacting a purified compound with a suitable organic or inorganic acid or base, as appropriate, and isolating the salt thus formed. Representative salts include the hydrobromide, hydrochloride, sulfate, bisulfate, nitrate, acetate, oxalate, palmitiate, stearate, laurate, borate, benzoate, lactate, phosphate, tosylate, besylate, esylate, citrate, maleate, fumarate, succinate, tartrate, naphthylate, mesylate, glucoheptonate, lactobionate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. Compounds having N-oxides of amino groups, such as produced by reaction with hydrogen peroxide, are also encompassed.

The term “selective” means that a ligand binds with greater affinity to a particular receptor when compared with the binding affinity of the ligand to another receptor. Preferably, the binding affinity of the ligand for the first receptor is about 50% or greater than the binding affinity for the second receptor. More preferably, the binding affinity of the ligand to the first receptor is about 75% or greater than the binding affinity to the second receptor. Most preferably, the binding affinity of the ligand to the first receptor is about 90% or greater than the binding affinity to the second receptor. It is contemplated that preferred compounds bind lipid mobilizing peptide receptors with micromolar or greater affinity. More preferred compounds bind lipid mobilizing peptide receptors with nanomolar or greater affinity. Preferred lipid mobilizing peptide receptor ligands of the present invention include compounds that are selective agonists of the lipid mobilizing peptide receptor. Lipid mobilizing peptide receptor ligands can be identified, for example, by screening a compound library. Methods of identifying agonists and antagonists of receptors are well known to those skilled in the art. In one embodiment, lipid mobilizing peptide receptor ligands can be identified by competition with a lipid mobilizing peptide in the human adipocyte lipolysis assay described below.

A “therapeutically effective amount” means an amount of a compound that, when administered to a subject for treating a disease, is sufficient to effect such treatment for the disease. The “therapeutically effective amount” will vary depending on the compound, the disease state being treated, the severity or the disease treated, the age and relative health of the subject, the route and form of administration, the judgment of the attending medical or veterinary practitioner, and other factors.

For purposes of the present invention, “treating” or “treatment” describes the management and care of a patient for the purpose of combating the disease, condition, or disorder. The terms embrace both preventative, i.e., prophylactic, and palliative treatment. Treating includes the administration of a compound of present invention to prevent the onset of the symptoms or complications, alleviating the symptoms or complications, or eliminating the disease, condition, or disorder. Treating obesity therefore includes the reduction of appetite, the inhibition of food intake, the inhibition of weight gain, and inducing weight loss in patients in need thereof.

The form in which the active compound is administered to the cell is not critical; the active compound need only reach the cell, directly or indirectly. The invention encompasses preparation and use of medicaments and pharmaceutical compositions comprising a compound described herein (e.g., a VLMP, a VLMP agonist, or a derivative or structural analog of one of these) as an active ingredient. A derivative compound of a vertebrate lipid mobilizing peptide is a compound in which at least one amino acid residue has been derivatized, has been substituted with a conservative amino acid substitution, has been substituted with a amino acid residue not encoded by the genetic code or has been substituted by a non-amino acid moiety.

The polypeptide compound is preferably highly purified prior to incorporating it into the pharmaceutical composition (e.g., purity of at least 75%, 80%, 90%, 95%, 98%, 99%, or nearly 100% pure, by weight of dry polypeptide in the purified sample).

A VLMP may contain asymmetric or chiral centers, and therefore, exist in different stereoisomeric forms. It is contemplated that all stereoisomeric forms as well as mixtures thereof, including racemic mixtures, form part of the present invention. In addition, the present invention contemplates all geometric and positional isomers. For example, if a compound contains a double bond, both the cis and trans forms, as well as mixtures, are contemplated. Mixtures of isomers, including stereoisomers can be separated into their individual isomers on the basis of their physical chemical differences by methods well know to those skilled in the art, such as by chromatography and/or fractional crystallization. Enantiomers can be separated by converting the enantiomeric mixture into a diasteromeric mixture by reaction with an appropriate optically active compound (e.g., alcohol), separating the diastereomers and converting (e.g., hydrolyzing) the individual diastereomers to the corresponding pure enantiomers. Also, some of the compounds of this invention may be atropisomers (e.g., substituted biaryls) and are considered as part of this invention.

A VLMP may exist in unsolvated as well as solvated forms with pharmaceutically acceptable solvents such as water, ethanol, and the like. The present invention contemplates and encompasses both the solvated and unsolvated forms.

It is also possible that a VLMP may exist indifferent tautomeric forms. All tautomers of a VLMP are contemplated. Those skilled in the art will recognize that compound names contained herein may be based on a particular tautomer of a compound. While the name for only a particular tautomer may be used, it is intended that all tautomers are encompassed by the name of the particular tautomer, and all tautomers are considered part of the present invention.

It is also intended that the invention disclosed herein encompass compounds that are synthesized in vitro using laboratory techniques, such as those well known to synthetic chemists; or synthesized using in vivo techniques, such as through metabolism, fermentation, digestion, and the like. It is also contemplated that compounds may be synthesized using a combination of in vitro and in vivo techniques.

The present invention also includes isotopically labeled compounds, which are identical to the non-isotopically labeled compounds, but for the fact that one or more atoms are replaced by an atom having an atomic mass or mass number different from the atomic mass or mass number usually found most abundantly in nature. Examples of isotopes that can be incorporated into compounds identified by the present invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, ¹³⁵I and ³⁶Cl, respectively. VLMPs, prodrugs thereof, and pharmaceutically acceptable salts of said ligands or of said prodrugs which contain the aforementioned isotopes and/or other isotopes of other atoms are within the scope of this invention. Certain isotopically labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., 2H, may afford certain therapeutic advantages resulting from greater metabolic stability, for example increased in vivo half-life or reduced dosage requirements and, hence, may be preferred in some circumstances. Isotopically labeled compounds can generally be prepared by substituting a readily available isotopically labeled reagent for a non-isotopically labeled reagent.

A VLMP is administered to a patient in a therapeutically effective amount. A VLMP can be administered alone or as part of a pharmaceutically acceptable composition. In addition, a compound or composition can be administered all at once, as for example, by a bolus injection, multiple times, such as by a series of tablets, or delivered substantially uniformly over a period of time, as for example, using transdermal delivery. It is also noted that the dose of the compound can be varied over time. A VLMP can be administered using an immediate release formulation, a controlled release formulation, or combinations thereof. The term “controlled release” includes sustained release, delayed release, and combinations thereof.

A pharmaceutical composition of the invention can be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient that would be administered to a patient or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the human treated and further depending upon the route by which the composition is to be administered. By way of example, the composition can comprise between 0.1% and 100% (w/w) active ingredient. A unit dose of a pharmaceutical composition of the invention will generally comprise from about 100 milligrams to about 2 grams of the active ingredient, and preferably comprises from about 200 milligrams to about 1.0 gram of the active ingredient.

In addition, a VLMP can be administered alone, in combination with other VLMPs, or with other pharmaceutically active compounds. The other pharmaceutically active compounds can be selected to treat the same disease as the VLMP or a different disease. In certain embodiments, additional pharmaceutically active compounds include beta adrenergic receptor agonists (which can act synergistically with the polypeptide compounds described herein), serotonin re-uptake inhibitors (i.e., to reduce appetite), fat uptake blockers (to inhibit lipogenesis and fat deposition), and decoupling agents (e.g., thyroxine receptor binding agents).

If the patient is to receive or is receiving multiple pharmaceutically active compounds, the compounds can be administered simultaneously or sequentially in any order. For example, in the case of tablets, the active compounds may be found in one tablet or in separate tablets, which can be administered at once or sequentially in any order. In addition, it should be recognized that the compositions can be different forms. For example, one or more compounds may be delivered via a tablet, while another is administered via injection or orally as a syrup.

Another aspect of the invention relates to a kit comprising a pharmaceutical composition of the invention and instructional material. Instructional material includes a publication, a recording, a diagram, or any other medium of expression which is used to communicate the usefulness of the pharmaceutical composition of the invention for one of the purposes set forth herein in a human. The instructional material can also, for example, describe an appropriate dose of the pharmaceutical composition of the invention. The instructional material of the kit of the invention can, for example, be affixed to a container which contains a pharmaceutical composition of the invention or be shipped together with a container which contains the pharmaceutical composition. Alternatively, the instructional material can be shipped separately from the container with the intention that the instructional material and the pharmaceutical composition be used cooperatively by the recipient.

The invention also includes a kit comprising a pharmaceutical composition of the invention and a delivery device for delivering the composition to a human. By way of example, the delivery device can be a squeezable spray bottle, a metered-dose spray bottle, an aerosol spray device, an atomizer, a dry powder delivery device, a self-propelling solvent/powder-dispensing device, a syringe, a needle, a tampon, or a dosage-measuring container. The kit can further comprise an instructional material as described herein, relates to combining separate pharmaceutical compositions in kit form.

For example, a kit may comprise two separate pharmaceutical compositions comprising respectively a first composition comprising a VLMP or a VLMP agonist and a pharmaceutically acceptable carrier; and composition comprising second pharmaceutically active compound and a pharmaceutically acceptable carrier. The kit also comprises a container for the separate compositions, such as a divided bottle or a divided foil packet. Additional examples of containers include syringes, boxes, bags, and the like. Typically, a kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of a kit is a blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and a sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen that the tablets or capsules so specified should be ingested. Another example of such a memory aid is a calendar printed on the card, e.g., as follows “First Week, Monday, Tuesday, . . . etc. . . . Second Week, Monday, Tuesday,” etc. Other variations of memory aids will be readily apparent. A “daily dose” can be a single tablet or capsule or several pills or capsules to be taken on a given day. Also, a daily dose of a VLMP composition can consist of one tablet or capsule, while a daily dose of the second compound can consist of several tablets or capsules and vice versa. The memory aid should reflect this and assist in correct administration.

In another embodiment of the present invention, a dispenser designed to dispense the daily doses one at a time in the order of their intended use is provided. Preferably, the dispenser is equipped with a memory aid, so as to further facilitate compliance with the dosage regimen. An example of such a memory aid is a mechanical counter, which indicates the number of daily doses that have been dispensed. Another example of such a memory aid is a battery-powered micro-chip memory coupled with a liquid crystal readout, or audible reminder signal which, for example, reads out the date that the last daily dose has been taken and/or reminds one when the next dose is to be taken.

A VLMP composition, optionally comprising other pharmaceutically active compounds, can be administered to a patient either orally, rectally, parenterally, (for example, intravenously, intramuscularly, or subcutaneously) intracistemally, intravaginally, intraperitoneally, intravesically, locally (for example, powders, ointments or drops), or as a buccal or nasal spray. Other contemplated formulations include projected nanoparticles, liposomal preparations, resealed erythrocytes containing the active ingredient, and immunologically-based formulations.

Parenteral administration of a pharmaceutical composition includes any route of administration characterized by physical breaching of a tissue of a human and administration of the pharmaceutical composition through the breach in the tissue. Parenteral administration thus includes administration of a pharmaceutical composition by injection of the composition, by application of the composition through a surgical incision, by application of the composition through a tissue-penetrating non-surgical wound, and the like. In particular, parenteral administration includes subcutaneous, intraperitoneal, intravenous, intraarterial, intramuscular, or intrasternal injection and intravenous, intraarterial, or kidney dialytic infusion techniques.

Compositions suitable for parenteral injection comprise the active ingredient combined with a pharmaceutically acceptable carrier such as physiologically acceptable sterile aqueous or nonaqueous solutions, dispersions, suspensions, or emulsions, or may comprise sterile powders for reconstitution into sterile injectable solutions or dispersions. Examples of suitable aqueous and nonaqueous carriers, diluents, solvents, or vehicles include water, isotonic saline, ethanol, polyols (propylene glycol, polyethylene glycol, glycerol, and the like), suitable mixtures thereof, triglycerides, including vegetable oils such as olive oil, or injectable organic esters such as ethyl oleate. Proper fluidity can be maintained, for example, by the use of a coating such as lecithin, by the maintenance of the required particle size in the case of dispersions, and/or by the use of surfactants. Such formulations can be prepared, packaged, or sold in a form suitable for bolus administration or for continuous administration. Injectable formulations can be prepared, packaged, or sold in unit dosage form, such as in ampules, in multi-dose containers containing a preservative, or in single-use devices for auto-injection or injection by a medical practitioner.

Formulations for parenteral administration include suspensions, solutions, emulsions in oily or aqueous vehicles, pastes, and implantable sustained-release or biodegradable formulations. Such formulations can further comprise one or more additional ingredients including suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition. The pharmaceutical compositions can be prepared, packaged, or sold in the form of a sterile injectable aqueous or oily suspension or solution. This suspension or solution can be formulated according to the known art, and can comprise, in addition to the active ingredient, additional ingredients such as the dispersing agents, wetting agents, or suspending agents described herein. Such sterile injectable formulations can be prepared using a non-toxic parenterally-acceptable diluent or solvent, such as water or 1,3-butanediol, for example. Other acceptable diluents and solvents include Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation can comprise pharmaceutically acceptable polymeric or hydrophobic materials such as an emulsion, an ion exchange resin, a sparingly soluble polymer, or a sparingly soluble salt.

These compositions may also contain adjuvants such as preserving, wetting, emulsifying, and/or dispersing agents. Prevention of microorganism contamination of the compositions can be accomplished by the addition of various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents capable of delaying absorption, for example, aluminum monostearate and/or gelatin.

Dosage forms can include solid or injectable implants or depots. In preferred embodiments, the implant comprises an effective amount of an active agent selected from the group consisting of a VLMP, a VLMP agonist and a VLMP antagonist and a biodegradable polymer. In preferred embodiments, a suitable biodegradable polymer can be selected from the group consisting of a polyaspartate, polyglutamate, poly(L-lactide), a poly(D,L-lactide), a poly(lactide-co-glycolide), a poly(ε-caprolactone), a polyanhydride, a poly(beta-hydroxy butyrate), a poly(ortho ester) and a polyphosphazene. In other embodiments, the implant comprises an effective amount of active agent and a silastic polymer. The implant provides the release of an effective amount of active agent for an extended period of about one week to several years.

Solid dosage forms for oral administration include capsules, tablets, powders, and granules. In such solid dosage forms, the active compound is admixed with at least one inert customary excipient (or carrier) such as sodium citrate or dicalcium phosphate or (a) fillers or extenders, as for example, starches, lactose, sucrose, mannitol, or silicic acid; (b) binders, as for example, carboxymethylcellulose, alginates, gelatin, polyvinylpyrrolidone, sucrose, or acacia; (c) humectants, as for example, glycerol; (d) disintegrating agents, as for example, agar-agar, calcium carbonate, potato or tapioca starch, alginic acid, certain complex silicates, or sodium carbonate; (e) solution retarders, as for example, paraffin; (f) absorption accelerators, as for example, quaternary ammonium compounds; (g) wetting agents, as for example, cetyl alcohol or glycerol monostearate; (h) adsorbents, as for example, kaolin or bentonite; and/or (i) lubricants, as for example, talc, calcium stearate, magnesium stearate, solid polyethylene glycols, sodium lauryl sulfate, or mixtures thereof. In the case of capsules and tablets, the dosage forms may also comprise buffering agents.

A tablet comprising the active ingredient can, for example, be made by compressing or molding the active ingredient, optionally with one or more additional ingredients. Compressed tablets can be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets can be made by molding, in a suitable device, a mixture of the active ingredient, a pharmaceutically acceptable carrier, and at least sufficient liquid to moisten the mixture. Pharmaceutically acceptable excipients used in the manufacture of tablets include inert diluents, granulating and disintegrating agents, binding agents, and lubricating agents. Known dispersing agents include potato starch and sodium starch glycolate. Known surface active agents include sodium lauryl sulfate. Known diluents include calcium carbonate, sodium carbonate, lactose, microcrystalline cellulose, calcium phosphate, calcium hydrogen phosphate, and sodium phosphate. Known granulating and disintegrating agents include corn starch and alginic acid. Known binding agents include gelatin, acacia, pre-gelatinized maize starch, polyvinylpyrrolidone, and hydroxypropyl methylcellulose. Known lubricating agents include magnesium stearate, stearic acid, silica, and talc.

Tablets can be non-coated or they can be coated using known methods to achieve delayed disintegration in the gastrointestinal tract of a human, thereby providing sustained release and absorption of the active ingredient. By way of example, a material such as glyceryl monostearate or glyceryl distearate can be used to coat tablets. Further by way of example, tablets can be coated using methods described in U.S. Pat. Nos. 4,256,108; 4,160,452; and 4,265,874 to form osmotically-controlled release tablets. Tablets can further comprise a sweetening agent, a flavoring agent, a coloring agent, a preservative, or some combination of these in order to provide pharmaceutically elegant and palatable preparation.

Solid dosage forms such as tablets, dragees, capsules, and granules can be prepared with coatings or shells, such as enteric coatings and others well known in the art. They may also contain opacifying agents, and can also be of such composition that they release the active compound or compounds in a delayed manner. Examples of embedding compositions that can be used are polymeric substances and waxes. The active compounds can also be in micro-encapsulated form, if appropriate, with one or more of the above-mentioned excipients.

Solid compositions of a similar type may also be used as fillers in soft or hard filled gelatin capsules using such excipients as lactose or milk sugar, as well as high molecular weight polyethylene glycols, and the like. Hard capsules comprising the active ingredient can be made using a physiologically degradable composition, such as gelatin. Such hard capsules comprise the active ingredient, and can further comprise additional ingredients including, for example, an inert solid diluent such as calcium carbonate, calcium phosphate, or kaolin. Soft gelatin capsules comprising the active ingredient can be made using a physiologically degradable composition, such as gelatin. Such soft capsules comprise the active ingredient, which can be mixed with water or an oil medium such as peanut oil, liquid paraffin, or olive oil.

Oral compositions can be made, using known technology, which specifically release orally-administered agents in the small or large intestines of a human patient. For example, formulations for delivery to the gastrointestinal system, including the colon, include enteric coated systems, based, e.g., on methacrylate copolymers such as poly(methacrylic acid, methyl methacrylate), which are only soluble at pH 6 and above, so that the polymer only begins to dissolve on entry into the small intestine. The site where such polymer formulations disintegrate is dependent on the rate of intestinal transit and the amount of polymer present. For example, a relatively thick polymer coating is used for delivery to the proximal colon (Hardy et al., 1987 Aliment. Pharmacol. Therap. 1:273-280). Polymers capable of providing site-specific colonic delivery can also be used, wherein the polymer relies on the bacterial flora of the large bowel to provide enzymatic degradation of the polymer coat and hence release of the drug. For example, azopolymers (U.S. Pat. No. 4,663,308), glycosides (Friend et al., 1984, J. Med. Chem. 27:261-268) and a variety of naturally available and modified polysaccharides (see PCT application PCT/GB89/00581) can be used in such formulations.

Pulsed release technology such as that described in U.S. Pat. No. 4,777,049 can also be used to administer the active agent to a specific location within the gastrointestinal tract. Such systems permit drug delivery at a predetermined time and can be used to deliver the active agent, optionally together with other additives that my alter the local microenvironment to promote agent stability and uptake, directly to the colon, without relying on external conditions other than the presence of water to provide in vivo release.

Liquid dosage forms for oral administration include pharmaceutically acceptable emulsions, solutions, suspensions, syrups, and elixirs. In addition to the active compounds, the liquid dosage form may contain inert diluents commonly used in the art, such as water or other solvents, isotonic saline, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, almond oil, arachis oil, coconut oil, cottonseed oil, groundnut oil, corn germ oil, olive oil, castor oil, sesame seed oil, MIGLYOL™, glycerol, fractionated vegetable oils, mineral oils such as liquid paraffin, tetrahydrofurfuryl alcohol, polyethylene glycols, fatty acid esters of sorbitan, or mixtures of these substances, and the like. Besides such inert diluents, the composition can also include adjuvants, such as wetting agents, emulsifying and suspending agents, demulcents, preservatives, buffers, salts, sweetening, flavoring, coloring and perfuming agents. Suspensions, in addition to the active compound, may contain suspending agents, as for example, ethoxylated isostearyl alcohols, polyoxyethylene sorbitol or sorbitan esters, microcrystalline cellulose, hydrogenated edible fats, sodium alginate, polyvinylpyrrolidone, gum tragacanth, gum acacia, agar-agar, and cellulose derivatives such as sodium carboxymethylcellulose, methylcellulose, hydroxypropylmethylcellulose, aluminum metahydroxide, bentonite, or mixtures of these substances, and the like. Liquid formulations of a pharmaceutical composition of the invention that are suitable for oral administration can be prepared, packaged, and sold either in liquid form or in the form of a dry product intended for reconstitution with water or another suitable vehicle prior to use.

Known dispersing or wetting agents include naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include lecithin and acacia. Known preservatives include methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents can be prepared in substantially the same manner as liquid suspensions, the primary difference being that the active ingredient is dissolved, rather than suspended in the solvent. Liquid solutions of the pharmaceutical composition of the invention can comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

In other embodiments, the pharmaceutical composition can be prepared as a nutraceutical, i.e., in the form of, or added to, a food (e.g., a processed item intended for direct consumption) or a foodstuff (e.g., an edible ingredient intended for incorporation into a food prior to ingestion). Examples of suitable foods include candies such as lollipops, baked goods such as crackers, breads, cookies, and snack cakes, whole, pureed, or mashed fruits and vegetables, beverages, and processed meat products. Examples of suitable foodstuffs include milled grains and sugars, spices and other seasonings, and syrups. The polypeptide compositions described herein are preferably not exposed to high cooking temperatures for extended periods of time, in order to minimize degradation of the compounds.

Compositions for rectal or vaginal administration can be prepared by mixing a VLMP and any additional compounds with suitable non-irritating excipients or carriers such as cocoa butter, polyethylene glycol or a suppository wax, which are solid at ordinary room temperature, but liquid at body temperature, and therefore, melt in the rectum or vaginal cavity and release the VLMP. Such a composition can be in the form of, for example, a suppository, a retention enema preparation, and a solution for rectal or colonic irrigation. Suppository formulations can further comprise various additional ingredients including antioxidants and preservatives. Retention enema preparations or solutions for rectal or colonic irrigation can be made by combining the active ingredient with a pharmaceutically acceptable liquid carrier. As is known in the art, enema preparations can be administered using, and can be packaged within, a delivery device adapted to the rectal anatomy of a human. Enema preparations can further comprise various additional ingredients including antioxidants and preservatives.

A pharmaceutical composition of the invention can be prepared, packaged, or sold in a formulation suitable for vaginal administration. Such a composition can be in the form of, for example, a suppository, an impregnated or coated vaginally-insertable material such as a tampon, a douche preparation, or a solution for vaginal irrigation.

Dosage forms for topical administration of a VLMP include ointments, powders, sprays and inhalants. The compounds are admixed under sterile conditions with a physiologically acceptable carrier, and any preservatives, buffers, and/or propellants that may be required. Formulations suitable for topical administration include liquid or semi-liquid preparations such as liniments, lotions, oil-in-water or water-in-oil emulsions such as creams, ointments or pastes, and solutions or suspensions. Topically-administrable formulations can, for example, comprise from about 0.1% to about 10% (w/w) active ingredient, although the concentration of the active ingredient can be as high as the solubility limit of the active ingredient in the solvent. Formulations for topical administration can further comprise one or more of the additional ingredients described herein.

Ophthalmic formulations, eye ointments, powders, and solutions are also contemplated as being within the scope of this invention. Such formulations can, for example, be in the form of eye drops including, for example, a 0.1-1.0% (w/w) solution or suspension of the active ingredient in an aqueous or oily liquid carrier. Such drops can further comprise buffering agents, salts, or one or more other of the additional ingredients described herein. In other embodiments, ophthalmalmically administrable formulations comprise the active ingredient in microcrystalline form or in a liposomal preparation.

A pharmaceutical composition of the invention can be prepared, packaged, or sold in a formulation suitable for pulmonary administration via the buccal cavity. Such a formulation can comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant can be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point below 65 degrees F. at atmospheric pressure. Generally the propellant can constitute 50 to 99.9% (w/w) of the composition, and the active ingredient can constitute 0.1 to 20% (w/w) of the composition. The propellant can further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery can also provide the active ingredient in the form of droplets of a solution or suspension. Such formulations can be prepared, packaged, or sold as aqueous or dilute alcoholic solutions or suspensions, optionally sterile, comprising the active ingredient, and can conveniently be administered using any nebulization or atomization device. Such formulations can further comprise one or more additional ingredients including a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention. Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares. Formulations suitable for nasal administration can, for example, comprise from about as little as 0.1% (w/w) and as much as 100% (w/w) of the active ingredient, and can further comprise one or more of the additional ingredients described herein.

A pharmaceutical composition of the invention can be prepared, packaged, or sold in a formulation suitable for buccal administration. Such formulations can, for example, be in the form of tablets or lozenges made using conventional methods, and can, for example, comprise 0.1 to 20% (w/w) active ingredient, the balance comprising an orally dissolvable or degradable composition and, optionally, one or more of the additional ingredients described herein. Alternately, formulations suitable for buccal administration can comprise a powder or an aerosolized or atomized solution or suspension comprising the active ingredient. Such powdered, aerosolized, or atomized formulations, when dispersed, preferably have an average particle or droplet size in the range from about 0.1 to about 200 nanometers, and can further comprise one or more of the additional ingredients described herein.

For parenteral administration in non-human animals, the compound or compounds may be prepared in the form of a paste or a pellet and administered as an implant, usually under the skin of the head or ear of the animal. Paste formulations can be prepared by dispersing a compound or compounds in pharmaceutically acceptable oil such as peanut oil, sesame oil, corn oil or the like. Pellets containing a therapeutically effective amount of a compound or compounds can be prepared by admixing the compound with a diluent such as a carbowax, carnauba wax, and the like, and a lubricant, such as magnesium or calcium stearate, can be added to improve the pelleting process. It is, of course, recognized that more than one pellet may be administered to an animal to achieve the desired dose level. Moreover, it has been found that such implants may also be administered periodically during the animal treatment period in order to maintain the proper active agent level in the animal's body.

The present invention relates to the use of VLMPs to treat obesity, diabetes, sexual dysfunction, atherosclerosis, insulin resistance, impaired glucose tolerance, hypercholesterolemia, or hypertrigylceridemia. The methods of treatment of the present invention can also include combination therapy where other pharmaceutically active compounds useful for the treatment of obesity or other diseases are used in combination with a VLMP. It is known that obese patients have higher incidences of certain diseases such as atherosclerosis, hypercholesterolemia, hypertrigylceridemia, hypertension, sexual dysfunction (including erectile dysfunction), insulin resistance, impaired glucose tolerance, diabetes, particularly non-insulin dependent diabetes mellitus (NIDDM or Type 2 diabetes) and the diseases associated with diabetes such as nephropathy, neuropathy, retinopathy, cardiomyopathy, cataracts, and polycystic ovary syndrome. These diseases can be treated indirectly by treating obesity using a VLMP or directly by treating the specific disease itself using a VLMP. These diseases can be treated in the absence of obesity using a VLMP.

In one embodiment of the invention, an obese patient or a patient at risk of becoming obese can be administered a composition comprising an active agent selected from the group consisting of a VLMP and a VLMP agonist, a pharmaceutically acceptable carrier and at least one compound useful to treat obesity, diabetes including (NIDDM) and the conditions and/or diseases associated with diabetes, such as nephropathy, neuropathy, retinopathy, cardiomyopathy, cataracts, and polycystic ovary syndrome, atherosclerosis, hypercholesterolemia, hypertrigylceridemia, sexual dysfunction (including erectile dysfunction), insulin resistance, or impaired glucose tolerance, or combinations of compounds useful to treat these diseases.

Sexual dysfunction occurs in males and females and includes hypoactive sexual desire disorder, sexual anhedonia and dyspareunia. Hypoactive sexual desire disorder is a disorder in which sexual fantasies and desire for sexual activity are persistently or recurrently diminished or absent, causing marked distress or interpersonal difficulties. Symptoms and signs of hypoactive sexual desire disorder include the patient complaining of a lack of interest in sex, even in ordinarily erotic situations. The disorder is usually associated with infrequent sexual activity, often causing serious conflict between partners. Sexual anhedonia is decreased or absent pleasure in sexual activity. Sexual anhedonia is almost always classified under hypoactive sexual desire disorder, because loss of pleasure typically results in loss of desire. Dyspareunia is painful coitus or attempted coitus.

Erectile dysfunction is another example of a sexual dysfunction. Erectile dysfunction, like obesity, is another condition that can result in severe emotional distress. Persons suffering from erectile dysfunction are unable to develop and/or maintain an erection of the penis. Historically, erectile dysfunction has been viewed as having biological and psychological components, and more effort appeared to be exerted on treating the psychological components of the condition. Only recently with the introduction of effective treatments have persons having this condition been offered an oral medicinal treatment.

Diabetes is found more frequently in obese patients than non-obese patients. In spite of the early discovery of insulin and its subsequent widespread use in the treatment of diabetes, and the later discovery of and use of sulfonylureas, biguanides and thiazolidenediones, such as troglitazone, rosiglitazone or pioglitazone, as oral hypoglycemic agents, the treatment of diabetes remains less than satisfactory.

The use of insulin currently requires multiple daily doses, usually by self-injection. Determination of the proper dosage of insulin requires frequent estimations of the sugar in urine or blood. The administration of an excess dose of insulin causes hypoglycemia, with effects ranging from mild abnormalities in blood glucose to coma, or even death. Treatment of non-insulin dependent diabetes mellitus (Type 2 diabetes, NIDDM) usually consists of a combination of diet, exercise, oral hypoglycemic agents, e.g., thiazolidenediones, and, in more severe cases, insulin. However, the clinically available hypoglycemic agents can have side effects that limit their use, or an agent may not be effective with a particular patient. In the case of insulin dependent diabetes mellitus (Type 1), insulin is usually the primary course of therapy. Additional hypoglycemic agents that have fewer side effects or succeed where others fail are needed.

Atherosclerosis, a disease of the arteries, is recognized to be a leading cause of death in the United States and Western Europe. The pathological sequence leading to atherosclerosis and occlusive heart disease is well known. The earliest stage in this sequence is the formation of “fatty streaks” in the carotid, coronary and cerebral arteries and in the aorta. These lesions are yellow in color due to the presence of lipid deposits found principally within smooth-muscle cells and in macrophages of the intima layer of the arteries and aorta. Further, it is postulated that most of the cholesterol found within the fatty streaks, in turn, give rise to development of “fibrous plaques,” which consist of accumulated intimal smooth muscle cells laden with lipid and are surrounded by extra-cellular lipid, collagen, elastin and proteoglycans. The cells plus matrix form a fibrous cap that covers a deeper deposit of cell debris and more extra-cellular lipid. The lipid is primarily free and esterified cholesterol. The fibrous plaque forms slowly, and is likely in time to become calcified and necrotic, advancing to a “complicated lesion,” which accounts for arterial occlusion and tendency toward mural thrombosis and arterial muscle spasm that characterize advanced atherosclerosis.

Epidemiological evidence has firmly established hyperlipidemia as a primary risk factor in causing cardiovascular disease (CVD) due to atherosclerosis. In recent years, leaders of the medical profession have placed renewed emphasis on lowering plasma cholesterol levels, and low density lipoprotein cholesterol in particular, as an essential step in prevention of CVD. The upper limits of “normal” are now known to be significantly lower than heretofore appreciated. As a result, large segments of Western populations are now realized to be at particularly high risk. Such independent risk factors include glucose intolerance, left ventricular hypertrophy, hypertension, and being of the male sex. Cardiovascular disease is especially prevalent among diabetic subjects, at least in part because of the existence of multiple independent risk factors in this population. Successful treatment of hyperlipidemia in the general population, and in diabetic subjects in particular, is therefore of exceptional medical importance.

Hypertension (or high blood pressure) is a condition that occurs in the human population as a secondary symptom to various other disorders such as renal artery stenosis, pheochromocytoma or endocrine disorders. However, hypertension is also evidenced in many patients in whom the causative agent or disorder is unknown. While such “essential” hypertension is often associated with disorders such as obesity, diabetes and hypertriglyceridemia, the relationship between these disorders has not been elucidated. Additionally, many patients display the symptoms of high blood pressure in the complete absence of any other signs of disease or disorder.

It is known that hypertension can directly lead to heart failure, renal failure and stroke (brain hemorrhaging). These conditions are capable of causing death in a patient. Hypertension can also contribute to the development of atherosclerosis and coronary disease. These conditions gradually weaken a patient and can lead to death.

The exact cause of essential hypertension is unknown, though a number of factors are believed to contribute to the onset of the disease. Among such factors are stress, uncontrolled emotions, unregulated hormone release (the renin, angiotensin, aldosterone system), excessive salt and water due to kidney malfunction, wall thickening and hypertrophy of the vasculature resulting in constricted blood vessels and genetic factors.

The treatment of essential hypertension has been undertaken bearing the foregoing factors in mind. Thus, a broad range of beta-blockers, vasoconstrictors, angiotensin converting enzyme inhibitors and the like have been developed and marketed as antihypertensives. The treatment of hypertension utilizing these compounds has proven beneficial in the prevention of short-interval deaths such as heart failure, renal failure and brain hemorrhaging.

Hypertension has been associated with elevated blood insulin levels, a condition known as hyperinsulinemia. Insulin, a peptide hormone whose primary actions are to promote glucose utilization, protein synthesis and the formation and storage of neutral lipids, also acts to promote vascular cell growth and increase renal sodium retention, among other things. These latter functions can be accomplished without affecting glucose levels and are known causes of hypertension. Peripheral vasculature growth, for example, can cause constriction of peripheral capillaries while sodium retention increases blood volume. Thus, the lowering of insulin levels in hyperinsulinemics can prevent abnormal vascular growth and renal sodium retention caused by high insulin levels and thereby alleviate hypertension.

A VLMP can be used in combination with one or more compounds that are useful to treat obesity. Examples of classes of compounds that can be used to treat obesity include the active compound(s) in appetite suppressants such as ADIPEX™, BONTRIL™, DESOXYN, GRADUMET™, FASTIN™, IONAMIN™, and MERIDIA™, and lipase inhibitors such as XENICAL™.

Additional anti-obesity agents that can be used in combination with a LMP include a β₃-adrenergic receptor agonist, a cholecystokinin-A agonist, a monoamine reuptake inhibitor, a sympathomimetic agent, a serotoninergic agent, a dopamine agonist, a melanocyte-stimulating hormone receptor agonist or mimetic, a melanocyte-stimulating hormone receptor analog, a cannabinoid receptor antagonist, a melanin concentrating hormone antagonist, leptin, a leptin analog, a leptin receptor agonist, a galanin antagonist, a bombesin agonist, a neuropeptide-Y antagonist (including NPY-1 and NPY-5), a thyromimetic agent, dehydroepiandrosterone or an analog thereof, a glucocorticoid receptor agonist or antagonist, an orexin receptor antagonist, a urocortin binding protein antagonist, a glucagon-like peptide-1 receptor agonist, and a ciliary neurotrophic factor.

Especially preferred anti-obesity agents that can be used in combination with a VLMP include compounds selected from the group consisting of sibutramine, fenfluramine, dexfenfluramine, bromocriptine, phentermine, orlistat, ephedrine, leptin, phenylpropanolamine, pseudoephedrine, {4-[2-(2-[6-aminopyridin-3-yl]-2(R)—-hydroxyethylamino)ethoxy]phenyl}acetic acid, {4-[2-(2-[6-aminopyridin-3-y-l]-2(R)-hydroxyethylamino)ethoxy]phenyl}benzoic acid, {4-[2-(2-[6-aminopyridin-3-yl]-2(R)-hydroxyethylamino)ethoxy]phenyl}propi-onic acid, and {4-[2-(2-[6-aminopyridin-3-yl]-2(R)-hydroxyethylamino)ethox-y]phenoxy}acetic acid.

Similarly, compounds that can be used to treat sexual dysfunction, and particularly erectile dysfunction, such as Viagra™ can also be used in combination with a VLMP. Other compounds that can be used to treat sexual dysfunction, particularly erectile dysfunction, and that can be used in combination with a LMP include apomorphine and IC351 (ICOS). A class of compounds that are useful to treat sexual dysfunction, particularly erectile dysfunction, are phophodiesterase V inhibitors. Examples of phosphodieserase V inhibitors can be found in U.S. Pat. No. 5,272,147.

In another aspect of the invention, a VLMP can be administered in combination with a compound that is known to treat hypertension. Examples of classes of compounds that can be used to treat hypertension include calcium blockers, ACE inhibitors, diuretics, angiotensin II receptor blockers, β-blockers, and α-adrenergic blockers. In addition, combinations of compounds in the above-recited classes have been used to treat hypertension. Some examples of specific compounds that can be used in combination with VLMPs include quinapril; amlodipine, including the besylate salt; nifedipine; doxazosin, including the mesylate salt; and prazosin, including the hydrochloride salt.

In another aspect, a VLMP can be used in combination with compounds useful for the treatment of diabetes, including impaired glucose tolerance, insulin resistance, insulin dependent diabetes mellitus (Type 1) and non-insulin dependent diabetes mellitus (NIDDM or Type 2). Also included in the treatment of diabetes are the diabetic complications, such as neuropathy, nephropathy, retinopathy, cardiomyopathy or cataracts.

Representative agents that can be used to treat diabetes and which can be used in combination with a VLMP include, but are not limited to, insulin and insulin analogs (e.g., LysPro insulin); GLP-1 (7-37) (insulinotropin) and GLP-1 (7-36)—NH₂; sulfonylureas and analogs: chlorpropamide, glibenclamide, tolbutamide, tolazamide, acetohexamide, glypizide, glimepiride, repaglinide, meglitinide; biguanides: metformin, phenformin, buformin; alpha2-antagonists and imidazolines: midaglizole, isaglidole, deriglidole, idazoxan, efaroxan, fluparoxan; other insulin secretagogues: linogliride, A-4166; glitazones: ciglitazone, pioglitazone, englitazone, troglitazone, darglitazone, BRL49653; fatty acid oxidation inhibitors: clomnoxir, etomoxir; alpha-glucosidase inhibitors: acarbose, miglitol, emiglitate, voglibose, MDL-25,637, camiglibose, MDL-73,945; β-agonists: BRL 35135, BRL 37344, Ro 16-8714, ICI D7114, CL 316,243; phosphodiesterase inhibitors: L-386,398; lipid-lowering agents: benfluorex; antiobesity agents: fenfluramine and orlistat; vanadate and vanadium complexes (e.g., NAGLIVAN™) and peroxovanadium complexes; amylin antagonists; glucagon antagonists; gluconeogenesis inhibitors; somatostatin agonists and antagonists; antilipolytic agents: nicotinic acid, acipimox, WAG 994; and glycogen phosphorylase inhibitors. Also contemplated in combination with compounds of the present invention are pramlintide acetate (SYMLIN™) and nateglinide.

Excess lipid storage is associated with a variety of undesirable conditions and disorders. For example, fat accumulation can cause cosmetically undesirable body shape and size, and can increase the incidence of various disorders. Examples of these disorders include obesity, overweight, type II diabetes, cholelithiasis, hypertension, coronary heart disease, arthritis, various cancers (e.g., breast, colorectal, and endometrial cancers), renal failure, liver disease, chronic pain (e.g., lower back pain), sleep apnea, stroke, and urinary incontinence. A patient afflicted with one or more of these conditions or disorders can use the compositions and methods described herein to alleviate, reverse, or eliminate the condition or disorder. A patient at risk for developing one of these conditions or disorders can use the compositions and methods described herein to inhibit or prevent its occurrence.

Atherosclerosis is a condition wherein deposits containing cholesterol, lipid materials, and lipid-laden macrophages accumulate on and in the intimal and inner medial layers of arteries. Prolonged or excessive atherosclerosis can lead to thickening and loss of elasticity of arterial walls, to chronic ischemic disorders, to chronic thrombotic disorders, or to combinations of these. The compositions and methods described herein can be used to inhibit or prevent development and growth of atherosclerotic deposits or to diminish the size or extent of existing deposits. Owing to this capability, the methods and compositions described herein can inhibit or alleviate conditions and disorders attributable, at least in part to atherosclerosis. Examples of these disorders include high blood pressure, coronary artery disease, cardiac insufficiency, and stroke. It is not necessary that atherosclerotic deposits be detected in a patient before administering a composition comprising a vertebrate VLMP to the patient. Instead, the composition can be administered as part of a normal diet, as part of a diet prescribed for a person who exhibits abnormally high systemic cholesterol or lipid levels, or to a patient who is believed for some other reason to be at risk for developing atherosclerosis. Without being bound by any particular theory of operation, it is believed that the compositions and methods described herein induce or enhance mobilization of lipids from lipid-laden macrophages, and that the compositions and methods can also induce or enhance lipolysis of lipid materials in atherosclerotic deposits.

In another aspect, the peptides of the present invention, the stereoisomers and prodrugs thereof, and the pharmaceutically acceptable salts of the compounds, stereoisomers and prodrugs, can be employed in combination with an anti-obesity agent.

The anti-obesity agent is preferably selected from the group consisting of an apolipoprotein-B secretion/microsomal triglyceride transfer protein (apo-B/MTP) inhibitor, an MCR-4 agonist, a cholecystokinin-A (CCK-A) agonist, a monoamine reuptake inhibitor (such as sibutramine), a sympathomimetic agent, a serotoninergic agent (such as fenfluramine or dexfenfluramine), a dopamine agonist (such as bromocriptine), a melanocyte-stimulating hormone receptor analog, a cannabinoid receptor antagonist, a melanin concentrating hormone antagonist, leptin (the OB protein), a leptin analog, a leptin receptor agonist, a galanin antagonist, a lipase inhibitor (such as tetrahydrolipstatin, i.e. orlistat), an anorectic agent (such as a bombesin agonist), a Neuropeptide-Y antagonist, a thyromimetic agent, dehydroepiandrosterone or an analog thereof, a glucocorticoid receptor agonist or antagonist, an orexin receptor antagonist, a urocortin binding protein antagonist, a glucagon-like peptide-1 receptor agonist, a ciliary neurotrophic factor (such as Axokine), and human agouti-related protein (AGRP). Other anti-obesity agents, including the preferred agents set forth hereinbelow, are well known, or will be readily apparent in light of the instant disclosure, to one of ordinary skill in the art. Preferred anti-obesity agents include a compound selected from the group consisting of orlistat, sibutramine, fenfluramine, dexfenfluramine, bromocriptine, phentermine, ephedrine, leptin, phenylpropanolamine, and pseudoephedrine.

Representative anti-obesity agents for use in the combinations, pharmaceutical compositions, and methods of the invention can be prepared using methods known to one of ordinary skill in the art, for example, phentermine can be prepared as described in U.S. Pat. No. 2,408,345; sibutramine can be prepared as described in U.S. Pat. No. 4,929,629; fenfluramine and dexfenfluramine can be prepared as described in U.S. Pat. No. 3,198,834; and bromocriptine can be prepared as described in U.S. Pat. Nos. 3,752,814 and 3,752,888; and orlistat can be prepared as described in U.S. Pat. Nos. 5,274,143; 5,420,305; 5,540,917 and 5,643,874.

The invention further provides methods of increasing the lean meat content in edible animals which methods comprise administering to the edible animal a lean meat increasing amount of the peptide of the present invention, a stereoisomer, or prodrug thereof, or a pharmaceutically acceptable salt of the compound, stereoisomer, or prodrug; a pharmaceutical composition comprising a lean meat increasing amount of a peptide of the present invention, a stereoisomer or prodrug thereof, or a pharmaceutically acceptable salt of the peptide, stereoisomer, or prodrug, and a pharmaceutically acceptable vehicle, carrier, or diluent; or a pharmaceutical composition comprising a lean meat increasing amount of a peptide of the present invention, a stereoisomer or prodrug thereof, or a pharmaceutically acceptable salt of the compound, stereoisomer, or prodrug, and a pharmaceutically acceptable vehicle, carrier, or diluent, and an anti-obesity agent.

The peptide of the present invention, the stereoisomers and prodrugs thereof, and the pharmaceutically acceptable salts of the peptides, stereoisomers, and prodrugs, can be administered to a patient at dosage levels in the range of from about 0.01 to about 1,000 mg per day. For a normal adult human having a body weight of about 70 kg, a dosage in the range of from about 0.01 to about 300 mg is typically sufficient. However, some variability in the general dosage range may be required depending upon the age and weight of the subject being treated, the intended route of administration, the particular anti-obesity agent being administered and the like. The determination of dosage ranges and optimal dosages for a particular patient is well within the ability of one of ordinary skill in the art having the benefit of the instant disclosure. It is also noted that the compounds of the present invention can be used in sustained release, controlled release, and delayed release formulations, which forms are also well known to one of ordinary skill in the art.

It is not critical whether the compound is administered directly to the cell, to a tissue comprising the cell, a body fluid that contacts the cell, or a body location from which the compound can diffuse or be transported to the cell. It is sufficient that the compound is administered to the patient in an amount and by a route whereby an amount of the compound sufficient to mobilize lipids in the cell arrives, directly or indirectly at the cell. The minimum amount varies with the identity of the VLMP. In some embodiments, the minimum amount is generally in the range from 10⁻⁹ to 10⁻⁵ molar. In other embodiments, the minimum amount is typically in the range from 10⁻⁷ to 10⁻⁵ molar.

In preferred embodiments, a pharmaceutical composition comprising a VLMP can be administered to a patient at dosage levels in the range of about 0.1 to about 7,000 mg per day. A preferred dosage range is about 1 to about 100 mg per day. In other embodiments, a pharmaceutical composition comprising a VLMP can be administered to deliver a dose of between 1 nanogram per day per kilogram body weight and 100 milligrams per day per kilogram body weight, preferably from about 0.1 to about 10 mg/kg body weight of the individual per day, and preferably to deliver of between 100 milligrams and 2 grams, to a human patient.

The specific dosage and dosage range that can be used depends on a number of factors, including the requirements of the patient, the severity of the condition or disease being treated, and the pharmacological activity of the compound being administered. The determination of dosage ranges and optimal dosages for a particular patient is well within the ordinary skill of one in the art in view of this disclosure. It is understood that the ordinarily skilled physician or veterinarian will readily determine and prescribe an effective amount of the compound to mobilize lipid stores, induce weight loss, or inhibit appetite in the patient. In so proceeding, the physician or veterinarian can, for example, prescribe a relatively low dose at first, subsequently increasing the dose until an appropriate response is obtained. It is further understood, however, that the specific dose level for any particular human will depend upon a variety of factors including the activity of the specific compound employed, the age, body weight, general health, gender, and diet of the human, the time of administration, the route of administration, the rate of excretion, any drug combination, and the severity of any disorder being treated.

In some embodiments, a peptide of the present invention, a stereoisomer or prodrug thereof, or a pharmaceutically acceptable salt of the stereoisomer or prodrug; or a peptide of the present invention, a stereoisomer or prodrug thereof is administered to a subject in need of treatment therewith, preferably in the form of a pharmaceutical composition. In other embodiments, a peptide of the present invention, or a pharmaceutically acceptable salt of the stereoisomer or prodrug and an anti-obesity agent is administered to a subject in need of treatment therewith, preferably in the form of a pharmaceutical composition. In the combination aspect of the invention, a peptide of the present invention, a stereoisomer or prodrug thereof, or a pharmaceutically acceptable salt of the stereoisomer or prodrug and the anti-obesity agent may be administered either separately or in a pharmaceutical composition comprising both. It is generally preferred that such administration be oral or pulmonary. However, if the subject being treated is unable to swallow, or oral administration is otherwise impaired or undesirable, parenteral or transdermal administration will be appropriate.

According to the methods of the invention, when the peptide of the present invention, a stereoisomer or prodrug thereof, or a pharmaceutically acceptable salt of the stereoisomer or prodrug; or a peptide of the present invention, a stereoisomer or prodrug thereof, or a pharmaceutically acceptable salt of the stereoisomer or prodrug and an anti-obesity agent are administered together, such administration can be sequential in time or simultaneous with the simultaneous method being generally preferred. For sequential administration, a peptide of the present invention, the stereoisomer or prodrug thereof, or the pharmaceutically acceptable salt of the stereoisomer or prodrug and the anti-obesity agent can be administered in any order. It is generally preferred that such administration be oral. When administered sequentially, the administration of each can be by the same or by different methods.

In some embodiments, the present invention includes peptides modified by conservative substitutions of selected amino acid residues.

In this regard, it is understood that amino acids may be substituted on the basis of side chain bulk, charge and/or hydrophobicity. Amino acid residues are classified into four major groups:

Acidic: The residue has a negative charge due to loss of H ion at physiological pH and the residue is attracted by aqueous solution so as to seek the surface positions in the conformation of a peptide in which it is contained when the peptide is in aqueous solution.

Basic: The residue has a positive charge due to association with H ion at physiological pH and the residue is attracted by aqueous solution so as to seek the surface positions in the conformation of a peptide in which it is contained when the peptide is in aqueous medium at physiological pH.

Neutral/non-polar: The residues are not charged at physiological pH and the residue is repelled by aqueous solution so as to seek the inner positions in the conformation of a peptide in which it is contained when the peptide is in aqueous medium. These residues are also designated “hydrophobic residues.”

Neutral/polar: The residues are not charged at physiological pH, but the residue is attracted by aqueous solution so as to seek the outer positions in the conformation of a peptide in which it is contained when the peptide is in aqueous medium.

Amino acid residues can be further classified as cyclic or non-cyclic, aromatic or non aromatic with respect to their side chain groups these designations being commonplace to the skilled artisan.

TABLE 3

| | | Preferred |
|---|---|---|
| Original | Exemplary Conservative | Conservative |
| Residue | Substitution | Substitution |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn | Asn |
| Glu | Asp | Asp |
| Gly | Pro | Pro |
| His | Asn, Gln, Lys, Arg | Arg |
| Ile | Leu, Val, Met, Ala | Leu |
| | Phe | |
| Leu | Ile, Val | Ile |
| | Met, Ala, Phe | |
| Lys | Arg, Gln, Asn | Arg |
| Met | Leu, Phe, Ile | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe | Leu |
| | Ala | |

In terms of variants between species, it should be noted that the human VLMP (SEQ ID NO: 14) is related to the mouse VLMP (SEQ ID NO: 16) by the replacement of Arg7 and Leu4 in the mouse peptide with Gln1 and Phe4 in the human peptide. See Table 3, above, and Table 6, below. Similarly, in the rat VLMP (SEQ ID NO: 15), Leu4 in the mouse peptide is replaced by Phe4 in the rat peptide.

Commonly encountered amino acids which are not encoded by the genetic code, include 2-amino adipic acid (Aad) for Glu and Asp; 2-aminopimelic acid (Apm) for Glu and Asp; 2-aminobutyric (Abu) acid for Met, Leu, and other aliphatic amino acids; 2-aminoheptanoic acid (Ahe) for Met, Leu and other aliphatic amino acids; 2-aminoisobutyric acid (Aib) for Gly; cyclohexylalanine (Cha) for Val, and Leu and Ile; homoarginine (Har) for Arg and Lys; 2,3-diaminopropionic acid (Dpr) for Lys, Arg and His; N-ethylglycine (EtGly) for Gly, Pro, and Ala; N-ethylglycine (EtGly) for Gly, Pro, and Ala; N-ethylasparagine (EtAsn) for Asn, and Gln; Hydroxyllysine (Hyl) for Lys; allohydroxyllysine (AHyl) for Lys; 3-(and 4)hydoxyproline (3Hyp, 4Hyp) for Pro, Ser, and Thr; allo-isoleucine (AIle) for Ile, Leu, and Val; ρ-amidinophenylalanine for Ala; N-methylglycine (MeGly, sarcosine) for Gly, Pro, and Ala; N-methylisoleucine (MeIle) for Ile; Norvaline (Nva) for Met and other aliphatic amino acids; Norleucine (Nle) for Met and other aliphatic amino acids; Ornithine (Orn) for Lys, Arg and His; Citrulline (Cit) and methionine sulfoxide (MSO) for Thr, Asn and Gln; N-methylphenylalanine (MePhe), trimethylphenylalanine, halo (F, Cl, Br, and I)phenylalanine, triflourylphenylalanine, for Phe.

In other embodiments, the following unusual or unnatural amino acid substitutions, singly or in combination, may be used: β-alanine, homoproline, hydroxyproline, L-3-(2′-naphthyl)-alanine, D-3-(2′-naphthyl)-alanine, 1-aminocyclopentanecarboxlic acid, sarcosine, β, thienyl-L-alanine, β-thienyl-D-alanine, D-3-(3-pyridyl)-alanine, aminoctanoic acid, aminocaproic acid, 7-aminoheptanoic acid, aminovaleric acid, S-acetamidomethyl-D-cysteine, S-acetamidomethyl-L-cysteine, t-butyl-D-cysteine, t-butyl-L-cysteine, S-ethyl-D-cysteine, S-ethyl-L-cysteine, L-aspartic acid(beta-benzyl ester), D-aspartic acid(β-benzyl ester), L-glutamic acid(gamma-benzyl ester), D-glutamic acid(gamma-benzyl ester), N-epsilon-2(2-chloro-CBZ)-L-lysine, N-epsilon-(2-chloro-CBZ)-D-lysine, N-epsilon-(CBZ)-L-lysine, N-epsilon-(CBZ)-D-lysine, p-chloro-D-phenylalanine, p-nitro-L-phenylalanine, L-serine (OBzl), D-serine(OBzl), D-threonine(OBzl), L-threonine(OBzl), O-(2,6-dichlorobenzyl)-L-tyrosine, O-t-butyl-L-tyrosine, and O-t-butyl-D-tyrosine.

A useful method for identification of certain residues or regions of a VLMP variant for amino acid substitution other than those described herein for receptor specificity is called alanine scanning mutagenesis as described by Cunningham and Wells (1989) Science, 244:1081-1085. Here a residue or group of target residues are identified (e.g. charged residues such as Arg, Asp, His, Lys, and Glu) and replaced by a neutral or negatively charged amino acid to affect the interaction of the amino acids with the surrounding aqueous environment in or outside the cell. Those domains demonstrating functional sensitivity to the substitution then are refined by introducing further or other variations at or for the sites of substitution. Thus while the site for introducing an amino acid sequence variation is predetermined the nature of the mutation per se need not be predetermined. For example, to optimize the performance of a mutation at a given site, Ala scanning or random mutagenesis may be conducted at the target codon or region and the expressed VLMP variants screened for the optimal combination of desired activity.

Phage display of protein or peptide libraries offers another methodology for the selection of VLMP variants with improved affinity, altered specificity, or improved stability (Smith, G. P., (1991) Curr. Opin. Biotechnol. 2:668-673). High affinity proteins, displayed in a monovalent fashion as fusions with the M13 gene III coat protein (Clackson, T., (1994) et al., Trends Biotechnol. 12:173-183), can be identified by cloning and sequencing the corresponding DNA packaged in the phagemid particles after a number of rounds of binding selection.

Other VLMP variants include fusions such as those described in International Publication No. WO97/20939 as well as C-terminal fusions with proteins having a long half-life such as immunoglobulin constant region or other immunoglobulin regions, albumin, or ferritin as described in WO 89/02922 published Apr. 6, 1989. As used herein, the term “immunoadhesin” designates antibody-like molecules which combine the “binding domain” of a heterologous protein (an “adhesin”) with the effector functions of immunoglobulin constant domains. Structurally, the immunoadhesins comprise a fusion of the adhesin amino acid sequence with the desired binding specificity which is other than the antigen recognition and binding site (antigen combining site) of an antibody (i.e. is “heterologous”) and an immunoglobulin constant domain sequence. The immunoglobulin constant domain sequence in the immunoadhesin may be obtained from any immunoglobulin, such as IgG₁, IgG₂, IgG₃, or IgG₄ subtypes, IgA, IgE, IgD or IgM. Immunoadhesins are described in, for example, U.S. Pat. No. 5,116,964.

Peptides as described above and herein may be prepared or modified to have unusual amino acid residues such that the resultant peptide has increased binding affinity and/or stability due to increased resistance to enzymatic degradation, and thus provide agonists or antagonists which possess higher activity and longer duration of activity. One or more of the naturally-occurring L-amino acid residues of the peptides described herein can be replaced with the corresponding D-isomeric form. For example, Lys and/or Arg residues in the peptides may be substituted with (D)-Lys and or (D)-Arg or another basic amino acid or nonbasic residue to confer greater plasma stability. Biologically active analogues of the above described peptide sequences are also included within the scope of this invention in which the stereochemistry of individual amino acids may be inverted from (L)/S to (D)/R at one or more specific sites. Also included within this category are analogues modified by glycosylations of Asn, Ser and/or Thr residues, or sterically constrained amino acids such as C-α-methyl-amino acids and N-α-methyl amino acids.

VLMPs can be composed of amino acids joined to each other by peptide bonds or modified peptide bonds, i.e., peptide isosteres, and may contain amino acids other than the 20 gene-encoded amino acids. The VLMPs may be modified by either natural processes, such as posttranslational processing, or by chemical modification techniques which are well known in the art. Such modifications are well described in basic texts and in more detailed monographs, as well as in a voluminous research literature. Modifications can occur anywhere in the VLMP, including the peptide backbone, the amino acid side-chains and the amino or carboxyl termini. It will be appreciated that the same type of modification may be present in the same or varying degrees at several sites in a given VLMP. Also, a given VLMP may contain many types of modifications. VLMPs may be branched, for example, as a result of ubiquitination, and they may be cyclic, with or without branching. Cyclic, branched, and branched cyclic VLMPs may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivatives covalent attachment of a lipid or lipid derivative, covalent attachment of phosphatidylinositol, cross-linking, cyclization, disulfide bond formation, demethylation, formation of covalent cross-links, formation of cysteine, formation of pyroglutamate, formulation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination.

The peptide compounds described herein can have amino acid residues which are modified without affecting biological activity. The amino and carboxyl termini can optionally be derivatized. If derivatized, the amino terminal residue can be a pyroglutamate residue. The pyroglutamate residue can be formed in situ from a glutamate or glutamine residue by glutamyl cyclase, an enzyme or group of enzymes that catalyzes conversion of a glutamate or glutamine residue to a pyroglutamate residue. Glutamyl cyclase is widely distributed in mammalian tissues, including, for example, brain, pituitary, spleen, thymus, and kidney tissues. Thus, cyclization of an amino-terminal glutamate residue can be achieved in vitro (e.g., by contacting the polypeptide compound with a commercial preparation of a glutamyl cyclase in the presence of appropriate reagents) or in vivo (e.g., by delivering the polypeptide compound to a tissue in which a glutamyl cyclase occurs).

Stability and efficacy can be improved by replacing the pyroglutamyl residue with alternative moieties known in the art, such as L-6-ketopiperidine-2-carbonyl-(Coppi, G., et al., Eur. J. Pharmacol. 1990 182: 185-188), (S)-4,5-dihydroorotic acid derivatives, such as 1-methyl-(S)-4,5-dihydroorotyl-(Suzuki, M., et al., J. Med. Chem. 1990 33:2130-2137, gamma-butyrolactone-gamma-carbonyl-, (Tanaka, K., Regul. Pept. 1992 38:129-133), L-pyro-2-aminoadipyl-(Tanaka, K., Regul. Pept. 1992 38:129-133), thioamide substituents (S for O) in pyroglutamtyl-(Lankiewicz, L., et al., Biochem. Biophys. Res. Commun. 1992 184: 359-366), alpha-((1S,2R))-2-methyl-4-oxocyclopentylcarbonyl-(Matsushita, M., Arzneimittelforschung. 1993 43: 813-817), (S)-2-oxoimidazoline-4-carbonyl-derivatives, such as 1-benzyl-(S)-2-oxoimidazoline-4-carbonyl-(Maeda, H., Int. J. Pept. Protein Res. 1989 33:403-411), sulphonamido derivative, such as (S)-isothioazolinidine-1,1-dioxie-3-carbonyl-(Brunetti, L., Faramaco. 2002 57:479-486), cyclopropane analogs such as 2,3-methano-pyroglutamyl-(Mapelli, C., et al., Biopolymers 1989 28:123-128), or (oxo-azetidinyl)carbonyl-(Mori, M., et al., Res. Commun. Chem. Pathol. Pharmacol. 1991 71:17-26). Similarly, the C-terminal amide can be replaced with a thioamide group (Alexandrova, M., et al., Endocrinol. Exp. 1987 21:43-49).

Optionally, the carboxyl terminal residue can be blocked with a carboxyl terminus blocking moiety, such as an amine (—NH₂) moiety. Alternatively, the carboxyl terminus can be blocked by formation at the terminus of an ester, ketone, or higher amide moiety. Examples of suitable carboxyl terminus blocking ester and ketone moieties include methyl, ethyl, and propyl moieties, and examples of suitable carboxyl terminus blocking higher amide moieties include mono- and di-alkylamino groups such as methylamino, ethylamino, dimethylamino, diethylamino, methylethylamino moieties. Carboxyl terminal amidation can be achieved in vivo, for example by delivering the polypeptide compound to a cell or tissue in which enzymes that catalyze alpha-carboxyl amidation occur. By way of example, conversion of a polypeptide compound having a carboxyl terminal glycine residue to a polypeptide compound wherein the glycine residue is replaced by an (—NH₂) moiety (i.e., a carboxyl terminal amidated polypeptide compound) is catalyzed by the bi-functional enzyme designated peptidylglycine alpha-amidating monooxygehase (Prigge et al., 2000, Cell. Mol. Life Sci. 57(8-9):1236-1259). Thus, the carboxyl terminus of a peptide compound having a carboxyl terminal glycine residue can be achieved by delivering the compound to a cell or tissue in which that bifunctional enzyme is expressed. Reagents and methods for producing these carboxyl group derivatives in vitro are known in the art. By way of example, polypeptide compounds can be amidated in vitro using any of a number of known carboxypeptidase or transamidase enzymes (e.g., as described in Aasmul-Olsen et al., 1991, Biomed. Biochim. Acta 50(10-11):S106-S109; Merkler, 1994, Enzyme Microb. Technol. 16(6):450-456) or using the bi-functional peptidylglycine alpha-amidating monooxygenase. Chemical methods of amidating carboxyl acid residues are known, and substantially any of those methods can be used to amidate the carboxyl terminus of the polypeptide compounds described herein.

The VLMP and analogs may be further modified to contain additional chemical moieties not normally part of the protein. Such chemically modified derivatives may provide additional advantages such as increased solubility, stability and circulating time of the polypeptide, or decreased immunogenicity (see U.S. Pat. Nos. 4,179,337; 5,342,940; 5,089,261 and 5,349,052). The chemical moieties for derivitization may be selected from water soluble polymers such as polyethylene glycol, ethylene glycol/propylene glycol copolymers, carboxymethylcellulose, dextran, polyvinyl alcohol and the like. The polypeptides may be modified at random positions within the molecule, or at predetermined positions within the molecule and may include one, two, three or more attached chemical moieties.

The polymer may be of any molecular weight, and may be branched or unbranched. For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog).

The polyethylene glycol molecules (or other chemical moieties) should be attached to the protein with consideration of effects on functional or antigenic domains of the protein. There are a number of attachment methods available to those skilled in the art, e.g., EP 0 401 384, herein incorporated by reference (coupling PEG to G-CSF), see also Malik et al., Exp. Hematol. 20:1028-1035 (1992) (reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues glutamic acid residues and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group.

One may specifically desire proteins chemically modified at the N-terminus. Using polyethylene glycol as an illustration of the present composition, one may select from a variety of polyethylene glycol molecules (by molecular weight, branching, etc.), the proportion of polyethylene glycol molecules to protein (polypeptide) molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective proteins chemically modified at the N-terminus modification may be accomplished by reductive alkylation which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer is achieved.

The VLMP can also be modified by the covalent attachment of a polyglutamate moiety. Conjugation to polyglutamate has been shown to improve the efficacy of chemotherapy agents such as paclitaxel.

The method by which the polypeptide compound is made or obtained is not critical. Polypeptide compounds that are useful in the compositions and methods described herein can be isolated from natural sources by methods known in the art or made synthetically or semi-synthetically.

As noted above, the term antibody includes intact molecules as well as antibody fragments (such as, for example, Fab and F(ab′)2 fragments) which are capable of specifically binding to protein. Fab and F(ab′)2 fragments lack the Fc fragment of intact antibody, clear more rapidly from the circulation, and may have less non-specific tissue binding than an intact antibody. Thus, these fragments are preferred, as well as the products of a Fab or other immunoglobulin expression library. Moreover, antibodies of the present invention include chimeric, single chain, and humanized antibodies.

The VLMPs described herein can be used to make antibody molecules (such as antibodies, single-chain antibodies, and antibody fragments comprising one or more antibody variable regions) that bind specifically with the polypeptide. Such antibodies can be used to purify the same VLMP or polypeptides that share an epitope with the VLMP from a suspension or solution. By way of example, an antibody that binds specifically to a VLMP can be used to isolate a polypeptide having a common epitope from a suspension prepared from a human, murine, bovine, porcine, or other mammalian or vertebrate cell or tissue sample. Other screening assays described herein can be used to assess whether the isolated polypeptide exhibits lipid mobilizing activity.

Antibodies can be prepared using any number of techniques known in the art. Suitable techniques are discussed briefly below. The antibody may be polyclonal or monoclonal. Polyclonal antibodies can have significant advantages for initial development, including rapidity of production and specificity for multiple epitopes, ensuring strong immunofluorescent staining and antigen capture. Monoclonal antibodies are adaptable to large-scale production; preferred embodiments include at least one monoclonal antibody specific for a VLMP. Because polyclonal preparations cannot be readily reproduced for large-scale production, another embodiment uses a cocktail of at least four monoclonal antibodies.

A single chain Fv (“scFv” or “sFv”) polypeptide is a covalently linked V_{H}:V_{L} heterodimer which may be expressed from a nucleic acid including V_{H}- and V_{L}-encoding sequences either joined directly or joined by a peptide-encoding linker. Huston, et al. Proc. Nat. Acad. Sci. USA, 85: 5879-5883 (1988). A number of structures for converting the naturally aggregated, but chemically separated, light and heavy polypeptide chains from an antibody V region into a scFv molecule which folds into a three dimensional structure substantially similar to the structure of an antigen-binding site. See, e.g. U.S. Pat. Nos. 6,512,097, 5,091,513 and 5,132,405 and 4,956,778.

In one class of embodiments, recombinant design methods can be used to develop suitable chemical structures (linkers) for converting two naturally associated, but chemically separate, heavy and light polypeptide chains from an antibody variable region into a sFv molecule which folds into a three-dimensional structure that is substantially similar to native antibody structure. Design criteria include determination of the appropriate length to span the distance between the C-terminal of one chain and the N-terminal of the other, wherein the linker is generally formed from small hydrophilic amino acid residues that do not tend to coil or form secondary structures. Such methods have been described in the art. See, e.g., U.S. Pat. Nos. 5,091,513 and 5,132,405 to Huston et al.; and U.S. Pat. No. 4,946,778 to Ladner et al.

In this regard, the first general step of linker design involves identification of plausible sites to be linked. Appropriate linkage sites on each of the V_{H} and V_{L} polypeptide domains include those which result in the minimum loss of residues from the polypeptide domains, and which necessitate a linker comprising a minimum number of residues consistent with the need for molecule stability. A pair of sites defines a “gap” to be linked. Linkers connecting the C-terminus of one domain to the N-terminus of the next generally comprise hydrophilic amino acids which assume an unstructured configuration in physiological solutions and preferably are free of residues having large side groups which might interfere with proper folding of the V_{H} and V_{L} chains. Thus, suitable linkers under the invention generally comprise polypeptide chains of alternating sets of glycine and serine residues, and may include glutamic acid and lysine residues inserted to enhance solubility. Nucleotide sequences encoding such linker moieties can be readily provided using various oligonucleotide synthesis techniques known in the art.

Alternatively, a humanized antibody fragment may comprise the antigen binding site of a murine monoclonal antibody and a variable region fragment (lacking the antigen binding site) derived from a human antibody. Procedures for the production of chimeric and further engineered monoclonal antibodies include those described in Riechmann et al. (Nature 332: 323,1988), Liu et al. (PNAS 84: 3439,1987), Larrick et al. (Bio/Technology 7: 934,1989), and Winter and Harris (TIPS 14: 139, May, 1993).

One method for producing a human antibody comprises immunizing a nonhuman animal, such as a transgenic mouse, with a VLMP, whereby antibodies directed against VLMP are generated in said animal. Procedures have been developed for generating human antibodies in non-human animals. The antibodies may be partially human, or preferably completely human. Non-human animals (such as transgenic mice) into which genetic material encoding one or more human immunoglobulin chains has been introduced may be employed. Such transgenic mice may be genetically altered in a variety of ways. The genetic manipulation may result in human immunoglobulin polypeptide chains replacing endogenous immunoglobulin chains in at least some (preferably virtually all) antibodies produced by the animal upon immunization. Antibodies produced by immunizing transgenic animals with a VLMP are provided herein.

Mice in which one or more endogenous immunoglobulin genes are inactivated by various means have been prepared. Human immunoglobulin genes have been introduced into the mice to replace the inactivated mouse genes. Antibodies produced in the animals incorporate human immunoglobulin polypeptide chains encoded by the human genetic material introduced into the animal. Examples of techniques for production and use of such transgenic animals are described in U.S. Pat. Nos. 5,814,318, 5,569,825, and 5,545,806, which are incorporated by reference herein.

Monoclonal antibodies may be produced by conventional procedures, e.g., by immortalizing spleen cells harvested from the transgenic animal after completion of the immunization schedule. The spleen cells may be fused with myeloma cells to produce hybridomas, by conventional procedures.

A method for producing a hybridoma cell line comprises immunizing such a transgenic animal with a immunogen comprising at least seven contiguous amino acid residues of a VLMP; harvesting spleen cells from the immunized animal; fusing the harvested spleen cells to a myeloma cell line, thereby generating hybridoma cells; and identifying a hybridoma cell line that produces a monoclonal antibody that binds a VLMP. Such hybridoma cell lines, and monoclonal antibodies produced therefrom, are encompassed by the present invention. Monoclonal antibodies secreted by the hybridoma cell line are purified by conventional techniques. In another embodiment, antibody fragments are produced by selection from a nonimmune phage display antibody repertoire against one set of antigens in the presence of a competing set of antigens (Stausbol-Grøn, B., et al., De novo identification of cell-type specific antibody-antigen pairs by phage display subtraction. Isolation of a human single chain antibody fragment against human keratin 14. Eur J Biochem 2001 May; 268(10):3099-107). This approach can be used to produce phage antibodies directed against VLMP antigens. The protocol in general is based on that described by Stausbol-Grøn, B., et al., 2001. Briefly, a nonimmunized semisynthetic phage display antibody repertoire is used. The repertoire is a single chain Fv (scFv) phagemid repertoire constructed by recloning the heavy and light chain regions from the lox library (Griffiths, A. D., et al. (1994) Isolation of high affinity human antibodies directly from large synthetic repertoires. EMBO J. 13, 3245-3260.). Escherichia coli TG1 (supE hsdD5 Δ(lac-proAB) thi F′{traD36proAB+lacI^{q} lacZΔM15]) is an amber suppressor strain (supE) and is used for propagation of phage particles. E. coli HB2151 (ara Δ(lac-proAB) thi F′{proAB+lacI^{q} lacZΔM15]) is a nonsuppressor strain and is used for expression of soluble scFv. In another embodiment, a human single-chain Fv (scFv) library can be amplified and rescued, as described (Gao, at al., Making chemistry selectable by linking it to infectivity, Proc. Natl. Acad. Sci. USA, Vol. 94, pp. 11777-11782, October 1997). The library is panned against VLMPs suspended in PBS (10 mM phosphate, 150 mM NaCl, pH 7.4) and the positive scFv-phage are selected by enzyme-linked immunosorbent assay (ELISA).

Antibodies may be employed in an in vitro procedure, or administered in vivo to inhibit biological activity induced by a VLMP. Disorders which may be caused or exacerbated (directly or indirectly) by the interaction of VLMPs of the present invention with cell surface receptors thus may be treated. A therapeutic method involves in vivo administration of a blocking antibody to a mammal in an amount effective for reducing a biological activity induced by a VLMP. Also provided herein are conjugates comprising a detectable (e.g., diagnostic) or therapeutic agent, attached to an antibody directed against a VLMP. Examples of such agents are well known, and include but are not limited to diagnostic radionuclides, therapeutic radionuclides, and cytotoxic drugs. See, e.g., Thrush et. al (Annu. Rev. Immunol., 14: 49-71,1996, p. 41). The conjugates find use in either in vitro or in vivo procedures.

Diagnostic and Prognostic Systems

A diagnostic system of the present invention in kit form includes, in an amount sufficient to perform at least one assay, a composition containing a polyclonal or monoclonal antibody of this invention or fragments thereof, as a separately packaged reagent. Instructions for use of the packaged reagent are also typically included. In another embodiment, the present invention provides prognostic methods of predicting the most appropriate therapeutic approach to optimize efficacy for a particular subject.

“Instructions for use” typically include a tangible expression describing the reagent concentration or at least one assay method parameter such as the relative amounts of reagent and sample to be admixed, maintenance time periods for reagent/sample admixtures, temperature, buffer conditions and the like. Also included, in one form or another, may be charts, graphs and the like that demonstrate predetermined concentration levels correlating specific physiological conditions to thrombotic events.

In preferred embodiments, the diagnostic system is used for assaying for the presence, and preferably amount, of a VLMP or fragment thereof in a body fluid sample, such as blood, plasma or urine according to the diagnostic methods described herein.

Preferably, the antibody is present as a monoclonal antibody composition, comprising a monoclonal antibody as described herein.

A diagnostic system of the present invention typically also includes a label or indicating means capable of signaling the formation of a specifically bound complex containing an antibody molecule of the present invention.

As used herein, the terms “label” and “indicating means” in their various grammatical forms refer to single atoms and molecules that are either directly or indirectly involved in the production of a detectable signal to indicate the presence of a complex. Any label or indicating means can be linked to or incorporated in an antibody molecule that is part of an antibody or monoclonal antibody composition of the present invention, or used separately, and those atoms or molecules can be used alone or in conjunction with additional reagents. Such labels are themselves well-known in clinical diagnostic chemistry and constitute a part of this invention only insofar as they are utilized with otherwise novel methods and/or systems.

The labeling means can be a fluorescent labeling agent that chemically binds to antibodies or antigens without denaturing them to form a fluorochrome (dye) that is a useful immunofluorescent tracer. Suitable fluorescent labeling agents are fluorochromes such as fluorescein isocyanate (FIC), fluorescein isothiocyante (FITC), 5-dimethylamine-1-naphthalenesulfonyl chloride (DANSC), tetramethylrhodamine isothiocyanate (TRITC), lissamine, rhodamine 8200 sulphonyl chloride (RB200 SC) and the like. A description of immunofluorescence analysis techniques is found in DeLuca “Immunofluorescence Analysis”, in Antibody As A Tool, Marchalonis et al., eds., John Wiley & Sons, Ltd., pp. 189-231 (1982) which is incorporated herein by reference.

In preferred embodiments, the indicating group is an enzyme, such as horseradish peroxidase (HRP), glucose oxidase, or the like. In such cases where the principal indicating group is an enzyme such as HRP or glucose oxidase, additional reagents are required to visualize the fact that a receptor-ligand complex (immunoreactant) has formed. Such additional reagents for HRP include hydrogen peroxide and an oxidation dye precursor such as diaminobenzidine. An additional reagent useful with glucose oxidase is 2,2′-azino-di-(3-ethyl-benzthiazoline-G-sulfonic acid) (ABTS).

Radioactive elements are also useful labeling agents and are used illustratively herein. An exemplary radiolabeling agent is a radioactive element that produces gamma ray emissions. Elements which themselves emit gamma rays, such as ¹²⁴I, ¹²⁵I, ¹²⁸I, ¹³²I and ⁵¹Cr represent one class of gamma ray emission-producing radioactive element indicating groups. Particularly preferred is ¹²⁵I. Another group of useful labeling means are those elements such as ¹¹C, ¹⁸F, ¹⁵O and ¹³N which themselves emit positrons. The positrons so emitted produce gamma rays upon encounters with electrons present in the animal's body. Also useful is a beta emitter, such as ¹¹¹In or ³H.

The linking of labels, i.e., labeling of, polypeptides and proteins is well known in the art. For instance, antibody molecules produced by a hybridoma can be labeled by metabolic incorporation of radioisotope-containing amino acids provided as a component in the culture medium. See, for example, Galfre et al., Meth. Enzymol., 73:3-46 (1981). The techniques of protein conjugation or coupling through activated functional groups are particularly applicable. See, for example, Avrameas, et al., Scand. J. Immunol., Vol. 8, Suppl. 7:7-23 (1978), Rodwell et al., Biotech., 3:889-894 (1984), and U.S. Pat. No. 4,493,795.

The diagnostic systems can also include, preferably as a separate package, a specific binding agent. A “specific binding agent” is a molecular entity capable of selectively binding a reagent species of the present invention but is not itself an antibody molecule of the present invention. Exemplary specific binding agents are antibody molecules, complement proteins or fragments thereof, protein A and the like. Preferably, the specific binding agent can bind the antibody molecule of this invention when it is present as part of a complex.

In preferred embodiments the specific binding agent is labeled. However, when the diagnostic system includes a specific binding agent that is not labeled, the agent is typically used as an amplifying means or reagent. In these embodiments, the labeled specific binding agent is capable of specifically binding the amplifying means when the amplifying means is bound to a reagent species-containing complex.

The diagnostic kits of the present invention can be used in an “ELISA” format to detect, for example, the presence or quantity of a VLMP or fragment thereof in a body fluid sample such as serum, plasma or urine. “ELISA” refers to an enzyme-linked immunosorbent assay that employs an antibody or antigen bound to a solid phase and an enzyme-antigen or enzyme-antibody conjugate to detect and quantify the amount of an antigen present in a sample. A description of the ELISA technique is found in Chapter 22 of the 4th Edition of Basic and Clinical Immunology by D. P. Sites et al., published by Lange Medical Publications of Los Altos, Calif. in 1982 and in U.S. Pat. No. 3,654,090; No. 3,850,752; and No. 4,016,043, which are all incorporated herein by reference.

Thus, in preferred embodiments, the antibody or antigen reagent component can be affixed (operatively linked) to a solid matrix to form a solid support that is separately packaged in the subject diagnostic systems. The reagent is typically affixed to the solid matrix by adsorption from an aqueous medium, although other modes of affixation, well known to those skilled in the art, can be used. Particularly preferred are embodiments suitable for competition ELISA assays wherein the antibody is in the liquid phase together with a sample containing an unknown amount of a VLMP or fragment thereof and the antigen is in the solid phase in an amount sufficient to compete with liquid phase antigen for immunoreaction with the liquid phase antibody. Useful solid matrices are well known in the art. Such materials include the cross-linked dextran available under the trademark SEPHADEX from Pharmacia Fine Chemicals (Piscataway, N.J.); agarose; polystyrene beads about 1 micron to about 5 millimeters in diameter available from Abbott Laboratories of North Chicago, Ill.; polyvinyl chloride, polystyrene, cross-linked polyacrylamide, nitrocellulose- or nylon-based webs such as sheets, strips or paddles; or tubes, plates or the wells of a microtiter plate such as those made from polystyrene or polyvinylchloride.

Thus, in particularly preferred embodiments, a diagnostic kit further contains, in a separate package, an antigen as described above for use in a the competitive ELISA assay in the form of a solid phase antigen as defined above.

The reagent species, labeled specific binding agent or amplifying reagent of any diagnostic system described herein can be provided in solution, as a liquid dispersion or as a substantially dry power, e.g., in lyophilized form. Where the indicating means is an enzyme, the enzyme's substrate can also be provided in a separate package of a system. A solid support such as the before-described microtiter plate and one or more buffers can also be included as separately packaged elements in this diagnostic assay system.

The packages discussed herein in relation to diagnostic systems are those customarily utilized in diagnostic systems. Such packages include glass and plastic (e.g., polyethylene, polypropylene and polycarbonate) bottles, vials, plastic and plastic-foil laminated envelopes and the like.

In preferred embodiments, the present invention provides diagnostic methods that result in detecting a VLMP or fragment thereof in a body fluid sample using an VLMP analog, a substantially isolated VLMP or fragment thereof or an antibody of this invention as a reagent to form an immunoreaction product whose amount relates, either directly or indirectly, to the presence, and preferably amount; of VLMP or fragment thereof in the sample.

Those skilled in the art will understand that there are numerous well known clinical diagnostic chemistry procedures in which an immunochemical reagent of this invention can be used to form an immunoreaction product whose amount relates to the amount of a VLMP or fragment thereof present in a body sample. Thus, while exemplary assay methods are described herein, the invention is not so limited. Various heterogeneous and homogeneous protocols, either competitive or noncompetitive, can be employed in performing an assay method of this invention.

Generally, to detect the presence of a VLMP or fragment thereof in a patient, an aliquot (i.e., a predetermined amount) of a body fluid sample, such as urine or a vascular fluid, namely blood, plasma or serum from the patient is contacted by admixture (admixed), with an antibody composition of the present invention to form an immunoreaction admixture. The admixture is then maintained under biological assay conditions for a period of time sufficient for the VLMP or fragment thereof present in the sample to immunoreact with (immunologically bind) a portion of the antibody combining sites present in the antibody composition to form a VLMP or VLMP fragment-antibody molecule immunoreaction product (immunocomplex). The complex can then be detected as described herein. The presence of the complex is indicative of a VLMP or a fragment thereof in the sample.

Maintenance time periods sufficient for immunoreaction are well known and are typically from about 10 minutes to about 16-20 hours at a temperature of about 4 degrees Celsius to about 45 degrees Celsius, with the time and temperature typically being inversely related. For example, longer maintenance times are utilized at lower temperatures, such as 16 hours at 4 degrees Celsius, and shorter times for higher temperatures, such as 1 hour at room temperature.

Biological assay conditions are those that maintain the biological activity of the immunochemical reagents of this invention and the VLMP or fragment thereof sought to be assayed such that the reagents retain their ability to form an immunoreaction product. Those conditions include a temperature range of about 4 degrees Celsius to about 45 degrees Celsius, a pH value of about 5 to about 9 and an ionic strength varying from that of distilled water to that of about one molar sodium chloride. Methods for optimizing such maintenance time periods and biological assay conditions are well known in the art.

In preferred embodiments, immunoassay is conducted using a cell free body fluid sample. By “cell free” is meant that the sample does not contain detectable amounts of cells, tissue or other macroscopic biological materials normally present in a body fluid such as blood. A body fluid sample typically contains cells as a normal component, or as a contaminant, and can be rendered cell-free by a variety of biochemical procedures including centrifugation, filtration and chromatography, so long as the retained fluid sample contains substantially all of the soluble protein initially present in the sample prior to removal of the cells. Determining the presence or amount of immunoreaction product formed by the above maintenance step, either directly or indirectly, can be accomplished by assay techniques well known in the art, and typically depend on the type of indicating means used.

In a preferred competition assay method, the immunoreaction admixture described above further contains a solid phase having affixed thereto a solid phase antigen comprising a VLMP analog or a polypeptide having an amino acid residue sequence that includes the sequence of a VLMP fragment of this invention. Thus, in this embodiment, the assay comprises the steps of: (a) admixing a body fluid sample with 1) an antibody composition of this invention and 2) a solid support having affixed thereto (operatively linked) a VLMP analog or a polypeptide having an amino acid residue sequence that includes the sequence of a VLMP fragment of this invention, or both, to form an immunoreaction admixture having both a liquid phase and a solid phase; (b) maintaining said immunoreaction admixture under biological assay conditions for a time period sufficient to form an immunoreaction product in the solid phase; and (c) detecting the presence, and preferably amount, of the immunoreaction product formed in the solid phase in step (b), and thereby the amount of presence/amount of a VLMP or fragment thereof in the body fluid sample.

Where a VLMP analog is used in the solid phase, the antibody composition contains antibody molecules that immunoreact with the VLMP analog. Preferably, the body fluid sample is a cell free body fluid sample such as urine or platelet poor plasma.

More preferably, detecting in step (c) is performed by the steps of: (i) admixing the immunoreaction product formed in step (b) with an indicating means to form a second reaction admixture; (ii) maintaining the second reaction admixture for a time period sufficient for said indication means to bind the immunoreaction product formed in step (b) and form a second reaction product; and, (iii) determining the presence and/or amount of indicating means in the second reaction product, and thereby the presence of the immunoreaction product formed in step (b). Particularly preferred is the use of a labeled second antibody, immunospecific for the first antibody, as the indicating means, and preferably the label is horseradish peroxidase.

In another competition assay format the immunoreaction admixture contains (1) a body fluid sample, preferably cell free, (2) an antibody of this invention and (3) a labeled VLMP analog or labeled VLMP fragment, wherein the antibody is present in the solid phase, being affixed to a solid support, to form a liquid and a solid phase. In this embodiment, the admixed body fluid sample competes with the labeled reagent for immunoreaction with the solid phase antibody to form a solid phase immunoreaction product. Thereafter, the detection of label in the solid phase correlates with the amount of a VLMP or fragment thereof in the admixed fluid sample.

Traditional polypeptide synthetic methods can be used to make the polypeptide compounds described herein. By way of example, traditional solid phase polypeptide synthetic methods using tert-butoxycarbonyl protecting groups; N-alpha-9-fluorenylmethoxycarbonyl protecting groups, or both, can be used to make the polypeptide compounds described herein.

In other embodiments, part or all of the peptide can be replaced by a peptide mimetic. Such peptide mimetics may include, for example, one or more of the following substitutions for —CO—NH— amide bonds: depsipeptides (—CO—O—), iminomethylenes (—CH₂—NH—), trans-alkenes (—CH═CH—), β-enaminonitriles (—C(═CH—CN)NH—), thioamides (—CS—NH—), thiomethylenes (—S—CH₂— or —CH₂—S), dimethylenes (—CH₂—CH₂-), ketomethylenes (COCH₂), N-meihyl peptides (CON(CH₃)) and retro-amides (—NH—CO—). Also included within this category are analogues modified by the insertion of a natural or unnatural amino acid into the peptide sequence. For instance, peptides are included which contain amino acid alkyl chains such as aminocaproic acid (Aca) within the sequence.

Assay

Two assay methods, giving equivalent results, can be used. The results disclosed in Example 2, below, were obtained using an in vitro assay based on measurement of glycerol released into the medium. An alternative assay system, based on the release of radioisotope from adipocytes incubated overnight with 9,10 ³H-palmitate, can also be used.

Cells

Human subcutaneous preadipocytes, obtained by liposuction (Zen-Bio, Inc. Research Triangle Park, N.C.), are cultured for 3 weeks in 96-well plates in Differentiation medium (DMEM/Hams F10 [1/1, v/v], 15 mM Hepes buffer, 3% FBS, Penicillin/Streptomycin, 33 uM Biotin 17 uM Pantothenate, 100 nM human insulin, 0.5 uM Dexamethasone, 0.2 mM IBMX, and 10 uM thiazolidinedione.

Glycerol Release Assay

The assay based on glycerol release is performed by removing the culture medium from differentiated cells, treating the cells with 0.1 ml of the test compounds at the appropriate doses in adipocyte medium (DMEM/Hank's F-10, 15 mM HEPES buffer, 3% FBS, penicillin/streptomycin) followed by incubation at 37 degrees Celsius for 2-5 hours. Isoproterenol is used as positive control and all assays are done in triplicate. At the end of the time points, 0.1 ml of the conditioned media from each well is transferred to a well of a new plate. Glycerol is quantified using standard enzymatic methods, with absorption by oxidized quinoneimine at 540 nm. Briefly, lipolytic activity can be assayed simply by the measurement of glycerol released into the medium since glycerokinase activity is not present in adipocytes (Carpene, C., Methods in Molecular Biology 155: Adipose Tissue Protocols, 1999). Glycerol released to the medium is phosphorylated by adenosine triphosphate (ATP) forming glycerol-1-phosphate (G-1-P) and adenosine-5′-diphosphate (ADP) in the reaction catalyzed by glycerol kinase. G-1-P is then oxidized by glycerol phosphate oxidase to dihydroxyacetone phosphate (DAP) and hydrogen peroxide (H₂O₂). A quinoneimine dye is produced by the peroxidase catalyzed coupling of 4-aminoantipyrine (4-AAP) and sodium N-ethyl-N-(3-sulfopropyl)ₘ-anisidine (ESPA) with H₂O₂, which shows an absorbance maximum at 540 nm. The increase in absorbance at 540 nm is directly proportional to glycerol concentration of the sample.

Isotope-Based Assay

The isotope-based assay starts with replacement of Adipocyte Growth Medium by Dulbecco's Modified Eagle's Medium (DMEM) containing 1 uCi/mL [9,10 ³H]-palmitate and 0.04% fatty acid free BSA. The following day the labeling medium is removed and the cells washed 3× with 0.1% fatty acid-free Bovine Serum Albumin (BSA) in Krebs-Ringers buffer. Test materials are added to 0.1% fatty acid-free BSA in Krebs-Ringer buffer to achieve desired concentration and incubated for 2-4 hours at 37 degrees Celsius. Isoproterenol is used as positive control. The assay is done in triplicate. Following incubation, 50 microliters of culture medium is removed from each well and scintillation counting is performed to detect released radioactive label. Total counts remaining in the well are also determined to calculate percentage of total label released.

### EXAMPLE 4

Identification of Vertebrate Lipid Mobilizing Peptides

Vertebrate lipid mobilizing peptides were identified by a search of databases using the following criteria developed based on the characteristics of insect adipokinetic hormones that are active in lipid mobilizing assays using human and mouse adipocytes, as disclosed in U.S. Published application No. U.S. 2003/0162717, the contents of which are incorporated by reference in their entirety. Based on the structure of the insect adipokinetic hormones, it was expected that vertebrate lipid mobilizing peptides would be relatively short, about 8 to 20 amino acid residues in length. It was further expected that vertebrate lipid mobilizing peptides would be secreted, and thus a mammalian secretion motif would be found upstream of the amino terminus of the active peptide.

The active insect adipokinetic hormones had been found to be “blocked” at both ends with a pyroglutamic acid residue at the amino terminus and an amide at the carboxyl terminus. Based on that observation, was expected that vertebrate lipid mobilizing peptide would share the same modifications. Thus, the amino terminal residue in the active peptide was expected to be glutamic acid (“Glu” in the three letter code or “E” in the one letter code) or glutamine (“Gln” or “Q”). Amidation is generally encoded by glycine (“Gly” or “G”) followed by one or two basic amino acid residues, most commonly lysine (“Lys” or “K”) and arginine (“Arg” or “R”). The other basic amino acid is histidine (“His” or “H”).

For the purpose of searching databases, the peptide sequence of the active form was filtered by a template (“AKH profile”) based on the frequency of amino acid residues in specific positions in a population of insect adipokinetic hormones, shown in Table 4, below. The search strategy also included that the peptide sequence of the active form would be within about 20 amino acids of a consensus secretion sequence.

TABLE 4
Peptide Search Template Based on Insect Adipokinetic Hormones

| Amino Acid | Position | | | | | | | | | | |
|---|---|---|---|---|---|---|---|---|---|---|---|
| Residue | Xaa1 | Xaa2 | Xaa3 | Xaa4 | Xaa5 | Xaa6 | Xaa7 | Xaa8 | Xaa9 | Xaa10 | Xaa11 |
| Glutamate or | 40 | | | | | | | | | | |
| Glutamine | | | | | | | | | | | |
| Leucine | | 24 | | | | | | | | | |
| Valine | | 11 | | | | | | | | | |
| Isoleucine | | 2 | | | | | | | | | |
| Phenylalanine | | 3 | | 36 | | | | | | | |
| Tyrosine | | 1 | | 4 | | | | 1 | | | |
| Asparagine | | | 27 | | | | 13 | | | 2 | |
| Threonine | | | 13 | | 19 | 6 | | | | 4 | |
| Serine | | | | | 20 | 4 | 6 | | | 1 | |
| Proline | | | | | | 29 | | | | | |
| Alanine | | | | | | 1 | | | | | |
| Glycine | | | | | | | | | 11 | 2 | |
| Tryptophan | | | | | | | 2 | 39 | | 1 | |
| Aspartic Acid | | | | | | | 5 | | | | |
| Valine | | | | | | | 1 | | | | |
| Lysine | | | | | | | | | | | 1 |

Initial analysis was performed on 191,089 records of human peptide sequences obtained from the GenBank™ database of the National Center for Biotechnology Information (NCBI). A search using an algorithm implemented in PERL of a profile based on the data presented in Table 4 (“AKH profile algorithm”) applied directly to the 191,089 records produced no results. The algorithm of von Jeijne (1986) was applied to prescreen the original 191,089 records producing a subset of 102,071 records. However, the application of the AKH profile algorithm to the prescreened subset of 102,071 records also produced no results.

In a further study, an algorithm implemented in PERL of a profile derived from the sequences of 415 human signal peptides 4 (“human signal peptide profile algorithm”) was applied to the 102,071 prescreened records, producing 65,178 records which were denoted as human peptides containing secretory signals. Application of the AKH profile algorithm to the 65,178 records of human peptides containing secretory signals identified 197 records.

In a further study, algorithms were applied to an updated and enlarged group of 195,092 records of human peptide sequences obtained from the GenBank™ database of the National Center for Biotechnology Information (NCBI). In this study, an algorithm implemented in PERL looked for a human signal peptide profile sequence and then looked for an AKH profile sequence “downstream” of the carboxyl terminus of the human signal peptide profile sequence (“forward algorithm”). A companion algorithm looked for an AKH profile sequence and then looked for a human signal peptide profile sequence “upstream” of the amino terminus of the AKH profile sequence (“backward algorithm”). The algorithms identified peptides using an AKH profile sequence that allowed for an amino terminal glutamine as well as an amino terminal glutamate. The backward and forward algorithms were verified by applying them to a fruit-fly genome database; an AKH peptide sequence was identified.

The benchmark set of 415 human signal peptide sequences fell within three standard deviations of the mean of the derived human signal peptide profile. Further criteria included exclusion of records having any of the following terms in the features of product or function fields: “enzyme”, “channel”, “transporter”, “cataly-,” and all words ending in “-ase.” Application of the backward and forward algorithms using the derived human signal peptide profile and the AKH peptide profile to the database of 195,092 records identified three GenBank records having a common sequence of signal peptide, linker and AKH profile sequence of IPELELPSSSPVPQVTTPEpgiyhgiclQLNFTASW (SEQ ID NO:1).

The first human match, XP_{—}044632.2, gi: 15318238 “similar to KIAA0556 protein [Homo sapiens]” 1669 aa (SEQ ID NO:2) is a sequence that was predicted by automated computational analysis by NCBI, and is derived from an annotated genomic sequence (NT_{—}010393) using gene prediction method: GNOMON, supported by mRNA and EST evidence. The second human match is T0330, gi: 7513017 “hypothetical protein KIAA0556-human (fragment)” 1081 aa (SEQ ID NO:3). The third human match is BAA25482.2, gi: 14133207 “KIAA0556 protein [Homo sapiens]” 1625 aa (SEQ ID NO:4).

A BLAST analysis in the Mammal subset of the nr database of NCBI identified thirteen matches with identities that were at least 60%. Three were the above human sequences. Nine other hits were in four records of mouse sequences, BAC35016.1, gi: 26342719, identities=72%, (SEQ ID NO: 5); AAH30902.1, gi:21410891, identities=90%, (SEQ ID NO: 6); BAC65591.3, gi:39104488, identities=94%, (SEQ ID NO: 7); BAC35027.1, gi:26342741, identities=63%, (SEQ ID NO: 8); and one match is a rat sequence, XP_{—}219359.2, gi: 34859171, identities=79%, (SEQ ID NO: 9).

A BLAST search of the entire nr database using the query IPELELPSSSPVPQVTTPEpgiyhgiclZLNFTASWGDLH (SEQ ID NO:10), where Z is either glutamate or glutamine, produced matches that are compared in alignments in Table 5, below.

TABLE 5
BLAST Alignments Of Mammalian 40 Mers

| Accession | ID/ | | | | SEQ |
|---|---|---|---|---|---|
| No. | Expect | Alignment | | | ID |
| XP_044632.3 | 39/40 | Query: 1 | IPELELPSSSPVPQVTTPEPGIYHGICLZLNFTASWGDLH | 40 | 10 |
| Human | 1e-30 | | IPELELPSSSPVPQVTTPEPGIYHGICL+LNFTASWGDLH | | |
| 1669 AA | | Sbjct: 1236 | IPELELPSSSPVPQVTTPEPGIYHGICLQLNFTASWGDLH | 1275 | 11 |
| BAA25482.2 | 39/40 | Query: 1 | IPELELPSSSPVPQVTTPEPGIYHGICLZLNFTASWGDLH | 40 | 10 |
| Human | 1e-30 | | IPELELPSSSPVPQVTTPEPGIYHGICL+LNFTASWGDLH | | |
| 1625 AA | | Sbjct: 1192 | IPELELPSSSPVPQVTTPEPGIYHGICLQLNFTASWGDLH | 1231 | 11 |
| T00330 | 39/40 | Query: 1 | IPELELPSSSPVPQVTTPEPGIYHGICLZLNFTASWGDLH | 40 | 10 |
| Human | 1e-30 | | IPELELPSSSPVPQVTTPEPGIYHGICL+LNFTASWGDLH | | |
| 1081 AA | | Sbjct: 648 | IFELELPSSSPVPQVTTPEPGIYHGICLQLNFTASWGDLH | 687 | 11 |
| BAC35016.1 | 28/40 | Query: 1 | IPELELPSSSPVPQVTTPEPGIYHGICLZLNFTASWGDLH | 40 | 10 |
| Mouse | 2e-16 | | +P LEL +S PV +VTTPEPGI++G+CL LN TASWGDLH | | |
| 965 AA | | Sbjct: 531 | VPGLELQTSPPVSEVTTPEPGIFYGLCLRLNLTASWGDLH | 570 | 12 |
| BAC35027.1 | 28/40 | Query: 1 | IPELELPSSSPVPQVTTPEPGIYHGICLZLNFTASWGDLH | 40 | 10 |
| Mouse | 2e-16 | | +P LEL +S PV +VTTPEPGI++G+CL LN TASWGDLH | | |
| 1019 AA | | Sbjct: 916 | VPGLELQTSPPVSEVTTPEPGIFYGLCLRLNLTASWGDLH | 955 | 12 |
| XP_355939.1 | 28/40 | Query: 1 | IPELELPSSSPVPQVTTPEPGIYHGICLZLNFTASWGDLH | 40 | 10 |
| Mouse | 2e-16 | | +P LEL +S PV +VTTPEPGI++G+CL LN TASWGDLH | | |
| 1621 AA | | Sbjct: 1187 | VPGLELQTSPPVSEVTTPEPGIFYGLCLRLNLTASWGDLH | 1226 | 12 |
| AAH30902.1 | 28/40 | Query: 1 | IPELELPSSSPVPQVTTPEPGIYHGICLZLNFTASWGDLH | 40 | 10 |
| Mouse | 2e-16 | | +P LEL +S PV +VTTPEPGI++G+CL LN TASWGDLH | | |
| 661 AA | | Sbjct: 227 | VPGLELQTSPPVSEVTTPEPGIFYGLCLRLNLTASWGDLH | 266 | 12 |
| BAC65591.3 | 28/40 | Query: 1 | IPELELPSSSPVPQVTTPEPGIYHGICLZLNFTASWGDLH | 40 | 10 |
| Mouse | 2e-16 | | +P LEL +S PV +VTTPEPGI++G+CL LN TASWGDLH | | |
| 532 AA | | Sbjct: 96 | VPGLELQTSPPVSEVTTPEPGIFYGLCLRLNLTASWGDLH | 135 | 12 |
| XP_341926.1 | 28/40 | Query: 1 | IPELELPSSSPVPQVTTPEPGIYHGICLZLNFTASWGDLH | 40 | 10 |
| Rat | 2e-15 | | +P LEL S PV V TPEPGI++G+CL LNFTASWGDLH | | |
| 1389 AA | | Sbjct: 1304 | VFGLELQSGPPVSDVFTPEPGIFYGLCLRLNFTASWGDLH | 1343 | 13 |

The mouse and rat nonapeptides identified by BLAST analysis (Table 6, below) have an arginine residue in the amino terminal position, a feature not found in any of the sequences of the known invertebrate AKH peptides (Table 4, above). The sequences of all three mammalian nonapeptides (SEQ ID NO: 14, 15 and 16) share another characteristic not found in any of the sequences of the known invertebrate AKH peptides, an alanine residue in position 6 (Xaa6) in combination with a serine residue in position 7 (Xaa7).

TABLE 6
Mammalian Lipid Mobilizing Peptides

| | | |
|---|---|---|
| Name | Sequence | SEQ ID |

| | | |
|---|---|---|
| Human | Gln-Leu-Asn-Phe-Thr-Ala-Ser-Trp-Gly | 14 |
| Rat | Arg-Leu-Asn-Phe-Thr-Ala-Ser-Trp-Gly | 15 |
| Mouse | Arg-Leu-Asn-Leu-Thr-Ala-Ser-Trp-Gly | 16 |

The matches listed in Table 5 above have high homology to the search template (SEQ ID NO: 10) as indicated by low “expect” scores in the range of 1×10⁻³⁰ to 2×10⁻¹⁵. All the GenBank sequences identified above in Table 5 are among those that have been partitioned into a non-redundant set of gene-oriented clusters related to the human KIAA0556 protein in the UniGene database of the National Center for Biotechnology Information. The human gene encodes the KIAA0556 protein, a hypothetical protein of unknown function that has been identified in silico (Nagase, T., et al., Prediction of the coding sequences of unidentified human genes. IX. The complete sequences of 100 new cDNA clones from brain which can code for large proteins in vitro, DNA Res. 5 (1), 31-39, 1998). Other vertebrate proteins, all of unknown function, that are members of the KIAA0556 gene cluster are listed in Table 7, below.

TABLE 7
Members of Human KIAA0556 UniGene Cluster

| | | Percent ID to T00330 |
|---|---|---|
| Vertebrate | UniGene Cluster ID | in Alignment Region* |
| Human | Hs.30512 | 100 |
| Mouse | D430042O09Rik | 81.2 |
| Rat | LOC361646 | 82.7 |
| Bovine | Bt.10058 | 87.5 |
| Pig | Ssc.6085 | 85.0 |
| Chicken | Gga.9001 | 76.7^{a} |
| Xenopus (frog) | XI.29814 | 79.3 |
| Zebrafish | Dr.16016 | 76.8 |
| Rainbow Trout | Omy.7157 | 59^{b} |

*KIAA0556 HomoloGene Entry in NCBI, Calculated Orthologs, except as noted below.
^{a}UniGene entry for Gga.9001
^{b}UniGene entry for Omy.7157

### EXAMPLE 2

Structure—Activity Relations of Vertebrate Lipid Mobilizing Peptides

All known invertebrate AKH peptides are characterized by both an amino terminal pyroglutamate residue and a blocked carboxyl terminal residue. Table 8 provides sequences of an active insect AKH octapeptide (SEQ ID NO: 17), an active insect AKH nonapeptide (SEQ ID NO: 23), an active insect AKH decapeptide (SEQ ID NO: 18) and an active insect HTP decapeptide (SEQ ID NO: 19), as well as three synthetic “alanine walk” analogs (SEQ ID NOs: 20-22) of the cockroach HTP peptide (SEQ ID NO: 19). All of the peptide having the native sequences (SEQ ID NOs: 17-19 and 23) were effective in lipid mobilization. However, the substitution of an alanine (SEQ ID NOs: 20-22) abolished measurable activity, even though these peptides each had an amino terminal pyroglutamate residue and a blocked carboxyl terminal residue.

TABLE 8
Structure-Activity Results: AKH Peptides

| | | |
|---|---|---|
| Name | Sequence | SEQ ID |

| | | |
|---|---|---|
| AKH II Schistocera | pGlu-Leu-Asn-Phe-Ser-Thr-Gly-TrpNH₂ | 17 |
| gregaria | | |
| AKH I Locusta | pGlu-Leu-Asn-Phe-Thr-Pro-Asn-Trp-Gly-ThrNH₂ | 18 |
| migratoria | | |
| HTP Blaberus | pGlu-Val-Asn-Phe-Ser-Pro-Gly-Trp-Gly-ThrNH₂ | 19 |
| discoidalis | | |
| Synthetic HTP analog 1 | pGlu-Val-Asn-Phe-Ser-Pro-Ala-Trp-Gly-ThrNH₂ | 20 |
| Synthetic HTP analog 2 | pGlu-Val-Asn-Phe-Ser-Pro-Gly-Ala-Gly-ThrNH₂ | 21 |
| Synthetic HTP analog 3 | pGlu-Val-Asn--Phe-Ser-Pro-Gly-Trp-Ala-ThrNH₂ | 22 |
| AKH Manduca sexta | pGlu-Leu-Thr-Phe-Thr-Ser-Ser-Trp-GlyNH₂ | 23 |
| or Heliothis zea | | |

Similarly, the ability to modulate lipid metabolism, as measured as glycerol releasing activity was studied for the peptides having the human amino acid sequence identified in Example 1 (SEQ ID NOs: 14 and 24), as well as derivatized analogs (SEQ ID NOs: 25, 26-30).

TABLE 9

Structure-Activity Study of Human
Lipid Mobilizing Peptides

| | | SEQ |
|---|---|---|
| Name | Sequence | ID |
| Peptide | Gln-Leu-Asn-Phe-Thr-Ala-Ser-Trp | 24 |
| 53 | | |
| Peptide | pGlu-Leu-Asn-Phe-Thr-Ala-Ser-Trp-Gly | 25 |
| 51 | | |
| Peptide | Gln-Leu-Asn-Phe-Thr-Ala-Ser-Trp-Gly | 14 |
| 52 | | |
| Peptide | Gln-Leu-Asn-Phe-Thr-Ala-Ser-Trp-GlyNH₂ | 26 |
| 72 | | |
| Peptide | pGlu-Leu-Asn-Phe-Thr-Ala-Ser-TrpNH₂ | 27 |
| 55 | | |
| Peptide | Gln-Leu-Asn-Phe-Thr-Ala-Ser-TrpNH₂ | 28 |
| 54 | | |
| Peptide | pGlu-Leu-Asn-Phe-Thr-Ala-Ser-Trp-GlyNH₂ | 29 |
| 73 | | |
| Peptide | pGlu-Leu-Asn-Phe-Thr-Ala-Ser-Trp | 30 |
| 60 | | |

Relative Activity of Human Analog Peptides, divided into four classes based on rank order of responses: 53,51>52,72>55,54>73,60

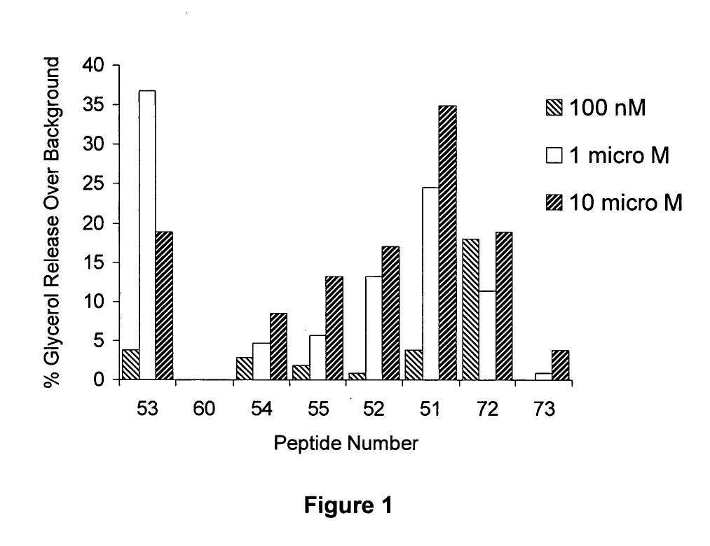

The peptides of Table 9 were tested in the glycerol release assay described above. The results are presented in FIG. 1 and in Table 10, below. FIG. 1 is a graphic illustration of the results of a study showing the modulation of the lipid metabolism of human cells by synthetic peptides having the identified human amino acid sequence (peptides 52 and 53) and derivatized analogues (peptides 51, 54, 55, 60, 72 and 73), as measured by the release of glycerol from human adipocytes in vitro at three concentrations: 100 nanomolar, 1 micromolar and 10 micromolar. The results showed that octamer and nonamer synthetic peptides having the sequence of the human peptide identified in Example 1 were effective in modulating the lipid metabolism of human cells as measured in this assay. Some derivatized analogs had effects comparable to the native sequences; compare peptides 51 and 53, peptides 72 and 52. In general, these peptides could be divided into four classes based on rank order of the glycerol releasing activity: 53, 51>52, 72>55, 54>73, 60.

An unexpected finding was that, unlike the insect AKH peptides, that human lipid mobilizing peptides were active without an amino terminal pyroglutamate residue or a blocked carboxyl terminal residue. In fact, those peptides having both an amino terminal pyroglutamate residue and blocked carboxyl terminal residue, peptides 55 and 73, showed low activity. Peptide 60, having an amino terminal pyroglutamate residue, showed no measurable activity in the assay.

TABLE 10
Structure-Activity Study of Human Lipid Mobilizing Peptides

| | | Percent Glycerol Release over Background | | | | | | | |
|---|---|---|---|---|---|---|---|---|---|
| Peptide | | 53 | 54 | 55 | 52 | 51 | 72 | 73 | Iso |
| 100 | nM | 3.8 | 2.8 | 1.9 | 0.9 | 3.8 | 18 | 0 | 213 |
| 1 | micro M | 36.8 | 4.7 | 5.7 | 13.2 | 24.5 | 11.3 | 0.9 | 262 |
| 10 | micro M | 18.9 | 8.5 | 13.2 | 17 | 34.9 | 18.9 | 3.8 | 206 |

The disclosure of every patent, patent application, and publication cited herein is hereby incorporated herein by reference in its entirety.

Although this invention has been disclosed with reference to specific embodiments, it is apparent that other embodiments and variations of this invention can be devised by others skilled in the art without departing from the true spirit and scope of the invention. The invention, as set forth in the appended claims, includes all such embodiments and equivalent variations. The claims should not be read as limited to the described order of elements unless stated to that effect.

1. An isolated vertebrate lipid mobilizing peptide encoded by a vertebrate gene, having the structure:
Xaa1-Xaa2-Xaa3-Xaa4-Xaa5-Xaa6-Xaa7-Xaa8-Xaa9-Xaa10-Xaa11 where Xaa1, Xaa2, Xaa3 and Xaa5 are any amino acid residue, Xaa4 is Phe or Leu, Xaa6 is a nonpolar amino acid residue, Xaa7 is an uncharged polar amino acid residue, Xaa9 is Gly or absent and Xaa10 and Xaa11 are present or absent, wherein the vertebrate lipid mobilizing peptide is translated operatively linked to a secretory signal sequence, and wherein the lipid metabolism of a vertebrate cell is modulated in response to contacting the cell with the vertebrate lipid mobilizing peptide.

2. The isolated vertebrate lipid mobilizing peptide of claim 1 wherein Xaa6 is Ala and Xaa7 is Ser.

3. The isolated vertebrate lipid mobilizing peptide of claim 1 wherein Xaa2 is Leu.

4. The isolated vertebrate lipid mobilizing peptide of claim 1 wherein Xaa3 is Asn.

5. The isolated vertebrate lipid mobilizing peptide of claim 1 wherein Xaa5 is Thr.

6. The isolated vertebrate lipid mobilizing peptide of claim 1 wherein Xaa8 is Trp.

7. An isolated vertebrate lipid mobilizing peptide encoded by a vertebrate KIAA0556 cluster gene, consisting essentially of 8-11 amino acid residues, wherein the vertebrate lipid mobilizing peptide is translated operatively linked to a secretory signal sequence, wherein the lipid metabolism of a vertebrate cell is; modulated in response to contacting the cell with the vertebrate lipid mobilizing peptide.

8. The isolated vertebrate lipid mobilizing peptide of claim 7 wherein the vertebrate KIAA0556 cluster gene is selected from the group consisting of Hs.30512, D430042O09Rik, LOC361646, Bt.10058, Ssc.6085, Gga.9001, Xl.29814, Dr.16016 and Omy.7157.

9. The isolated vertebrate lipid mobilizing peptide of claim 7 having the structure: Xaa1-Xaa2-Xaa3-Xaa4-Xaa5-Xaa6-Xaa7-Xaa8-Xaa9-Xaa10-Xaa11 where Xaa1, Xaa2, Xaa3 and Xaa5 are any amino acid residue, Xaa4 is Phe or Leu, Xaa6 is a nonpolar amino acid residue, Xaa7 is an uncharged polar amino acid residue, Xaa9 is Gly or absent and Xaa10 and Xaa11 are present or absent.

10. The isolated vertebrate lipid mobilizing peptide of claim 9 wherein Xaa6 is Ala and Xaa7 is Ser.

11. The isolated vertebrate lipid mobilizing peptide of claim 9 wherein Xaa2 is Leu.

12. The isolated vertebrate lipid mobilizing peptide of claim 9 wherein Xaa3 is Asn.

13. The isolated vertebrate lipid mobilizing peptide of claim 9 wherein Xaa5 is Thr.

14. The isolated vertebrate lipid mobilizing peptide of claim 9 wherein Xaa8 is Trp.

15. A derivative compound of the isolated vertebrate lipid mobilizing peptide of claim 9 wherein Xaa1 is selected from the group consisting of Gln, Arg, pGlu, and a pyroglutamyl alternative moiety.

16. The derivative compound of claim 15 wherein the pyroglutamyl alternative moiety selected from the group consisting of L-6-ketopiperidine-2-carbonyl-, (S)-4,5-dihydroorotic acid derivatives, gamma-butyrolactone-gamma-carbonyl-, L-pyro-2-aminoadipyl-, alpha-((1S,2R))-2-methyl-4-oxocyclopentylcarbonyl- and (S)-2-oxoimidazoline-4-carbonyl-derivatives.

17. A derivative compound of the isolated vertebrate lipid mobilizing peptide of claim 9 wherein at least one of the amino acid residues is derivatized.

18. A derivative compound of the isolated vertebrate lipid mobilizing peptide of claim 9 wherein at least one of the amino acid residues is conservatively substituted.

19. A derivative compound of the isolated vertebrate lipid mobilizing peptide of claim 9 wherein at least one of the amino acid residues is replaced by an amino acid not encoded by the genetic code.

20. A composition comprising the vertebrate lipid mobilizing peptide of claim 1 and a pharmaceutically acceptable carrier.

21. A kit comprising the vertebrate lipid mobilizing peptide of claim 1 and instructions for use.

22. A vertebrate lipid mobilizing peptide having the structure Xaa1-Leu-Asn-Xaa4-Thr-Ala-Ser-Trp-Xaa9-Xaa10-Xaa11, wherein Xaa1 is selected from the group consisting of Gln, Arg, and pGlu, Xaa4 is Phe or Leu, Xaa9 is Gly or absent and Xaa10 and Xaa11 are present or absent.

23. An isolated vertebrate lipid mobilizing peptide having a sequence selected from the group consisting of SEQ ID NOs:31, 32, 33, 34, 35, 36, 37 and 38.

24. A derivative compound of the vertebrate lipid mobilizing peptide of claim 22 where Xaa1 is a pyroglutamyl alternative moiety selected from the group consisting of L-6-ketopiperidine-2-carbonyl-, (S)-4,5-dihydroorotic acid derivatives, gamma-butyrolactone-gamma-carbonyl-, L-pyro-2-aminoadipyl-, alpha-((1S,2R))-2-methyl-4-oxocyclopentylcarbonyl- and (S)-2-oxoimidazoline-4-carbonyl-derivatives.

25. A derivative compound of the isolated vertebrate lipid mobilizing peptide of claim 22 wherein at least one of the amino acid residues is derivatized.

26. A derivative compound of the isolated vertebrate lipid mobilizing peptide of claim 22 wherein at least one of the amino acid residues is conservatively substituted.

27. A derivative compound of the isolated vertebrate lipid mobilizing peptide of claim 22 wherein at least one of the amino acid residues is replaced by an amino acid not encoded by the genetic code.

28. A composition comprising the vertebrate lipid mobilizing peptide of claim 22 and a pharmaceutically acceptable carrier.

29. A composition comprising the derivative compound of claim 17 and a pharmaceutically acceptable carrier.

30. A composition comprising the derivative compound of claim 25 and a pharmaceutically acceptable carrier.

31. A kit comprising the vertebrate lipid mobilizing peptide of claim 25 and instructions for use.

32. An isolated peptide having a sequence selected from the group consisting of SEQ ID NO:11, SEQ ID NO:12 and SEQ ID NO:13.

33. An isolated peptide having a sequence selected from the group consisting of SEQ ID NO:14, SEQ ID NO:15, SEQ ID NO:16, SEQ ID NO:24, SEQ ID NO:25, SEQ ID NO:26, SEQ ID NO:27, SEQ ID NO:28 and SEQ ID NO:29.

34. A composition comprising the isolated peptide of claim 32 and a pharmaceutically acceptable carrier.

35. A composition comprising the isolated peptide of claim 33 and a pharmaceutically acceptable carrier.

36. A kit comprising the composition of claim 34 and instructions for use.

37. A kit comprising the composition of claim 35 and instructions for use.

38. A vector comprising a nucleotide sequence encoding the peptide of claim 32.

39. A vector comprising a nucleotide sequence encoding the peptide of claim 33.

40. A cell comprising the vector of claim 38.

41. A cell comprising the vector of claim 39.

42. An antibody immunoreactive to the peptide of claim 1.

43. An antibody immunoreactive to the peptide of claim 7.

44. An antibody immunoreactive to the peptide of claim 22.

45. A diagnostic system of the present invention in kit form comprising a composition comprising a carrier and the antibody of claim 42 or fragments thereof in an amount sufficient to perform at least one assay as a separately packaged reagent and instructions for use of the packaged reagent.

46. A diagnostic system of the present invention in kit form comprising a composition comprising a carrier and the antibody of claim 43 or fragments thereof in an amount sufficient to perform at least one assay as a separately packaged reagent and instructions for use of the packaged reagent.

47. A diagnostic system of the present invention in kit form comprising a composition comprising a carrier and the antibody of claim 44 or fragments thereof in an amount sufficient to perform at least one assay as a separately packaged reagent and instructions for use of the packaged reagent.

48. A method of modulating lipid metabolism of a vertebrate subject comprising the step of administering an effective amount of the peptide of claim 1 or a derivative compound thereof.

49. A method of modulating lipid metabolism of a vertebrate subject comprising the step of administering an effective amount of the peptide of claim 7 or a derivative compound thereof.

50. A method of modulating lipid metabolism of a vertebrate subject comprising the step of administering an effective amount of the peptide of claim 22 or a derivative compound thereof.

51. A method of reducing the body fat of a vertebrate subject comprising the step of administering an effective amount of the peptide of claim 1 or a derivative compound thereof.

52. A method of reducing the body fat of a vertebrate subject comprising the step of administering an effective amount of the peptide of claim 7 or a derivative compound thereof.

53. A method of reducing the body fat of a vertebrate subject comprising the step of administering an effective amount of the peptide of claim 22 or a derivative compound thereof.

54. A method of reducing the body mass index of a human subject comprising the step of administering an effective amount of the peptide of claim 1 or a derivative compound thereof.

55. A method of reducing the body mass index of a human subject comprising the step of administering an effective amount of the peptide of claim 7 or a derivative compound thereof.

56. A method of reducing the body mass index of a human subject comprising the step of administering an effective amount of the peptide of claim 22 or a derivative compound thereof.
